(19) United States
(12) Patent Application Publication
(10) Pub. No.: US 2023/0398194 A1
(43) Pub. Date: Dec. 14, 2023

(54) METHODS AND COMPOSITIONS FOR ATTENUATING ANTIBIOTIC RESISTANCE

(71) Applicant: Theriva Biologics, Inc., Rockville, MD (US)

(72) Inventors: Sheila CONNELLY, Rockville, MD (US); Michael KALEKO, Rockville, MD (US)

(21) Appl. No.: 18/323,578

(22) Filed: May 25, 2023

Related U.S. Application Data
(63) Continuation of application No. 16/346,451, filed on Apr. 30, 2019.
(60) Provisional application No. 62/459,092, filed on Feb. 15, 2017.
(60) Provisional application No. 62/415,679, filed on Nov. 1, 2016.

(51) Int. Cl. A61K 38/50; A61K 31/407; A61K 31/43; A61K 31/546; A61K 38/14; C12Q 1/6876; A61K 31/545; A61K 35/74; C12N 9/86; A61P 1/00

(52) U.S. Cl. CPC A61K 38/50; A61K 31/407; A61K 31/43; A61K 31/546; A61K 38/14; C12Q 1/6876; C12Y 305/02006; A61K 31/545; A61K 35/74; C12N 9/86; A61P 1/00; C12Q 2600/106; C12Q 2600/158

(57) ABSTRACT

The present invention provides, in part, therapeutic beta-lactamases that can, inter alia, mitigate antibiotic resistance.

### CROSS-REFERENCE TO RELATED APPLICATIONS

This application claims the benefit of U.S. Provisional Application No. 62/415,679, filed Nov. 1, 2016 and U.S. Provisional Application No. 62/459,092, filed Feb. 15, 2017, the contents of each are hereby incorporated by reference herein in their entireties.

### FIELD

The present invention provides, in part, compositions and methods for mitigating antibiotic resistance with beta-lactamases.

### SEQUENCE LISTING

The instant application contains a Sequence Listing which has been submitted in ASCII format via EFS-Web and is hereby incorporated by reference in its entirety. The ASCII copy, created about Oct. 31, 2017, is 11.8 KB in size and is named SYN-028PC_ST25.txt.

### BACKGROUND

Antibiotic resistance occurs when bacteria change in a way that reduces the effectiveness of drugs, chemicals, or other agents designed to cure or prevent infections, including antibiotics. The resultant resistant bacteria survive and continue to multiply, therefore avoiding antibiotic treatment.

According to the CDC, each year in the United States, at least 2 million people become infected with bacteria that are resistant to antibiotics and at least 23,000 people die each year as a direct result of these infections.

There is an urgent and currently unmet medical need for compositions and methods to avoid or mitigate bacterial resistance to antibiotics.

### SUMMARY

Accordingly, the present invention provides, in some aspects, compositions and methods for allowing antibiotic administration to a subject without promoting, or by preventing or mitigating, antibiotic resistance. In various aspects, the present invention relates to the use of therapeutic beta-lactamases to modulate the microbiome, including the gastrointestinal (GI) microbiome, to reduce or prevent resistance to antibiotics. In various embodiments, the present invention relates to the use of therapeutic beta-lactamases to preserve a resistome state of a subject before administration of an antibiotic, e.g., to reduce or prevent an increased resistome presence. In various embodiments, the present invention relates to the use of therapeutic beta-lactamases to reduce a resistome state of a subject despite administration of an antibiotic.

In various aspects, the present invention relates to a method for treating or preventing an infection in a patient determined to be resistant to an antibiotic by administering an effective amount of a beta-lactamase having an amino acid sequence of at least 95% (or 97%, or 98%, or 99%, or 100%) identity with SEQ ID NO: 1 (and optionally having an Ambler D276N mutation) before or concurrently with the antibiotic or a different antibiotic.

In various embodiments, the patient is resistant to the antibiotic and the beta-lactamase provides a therapeutic response to the same antibiotic or the patient is resistant to the antibiotic and the beta-lactamase provides a therapeutic response to the different antibiotic.

In various embodiments, resistance to an antibiotic is determined using an antimicrobial susceptibility test (AST) or one or more sequencing methods (e.g., by determining by detecting the presence, absence, or level of one or more genes associated with resistance in a biological sample (e.g., stool or an aspirate of GI tract fluid) from the patient.

In various embodiments, the antibiotic resistance gene is one of the genes listed in Table A. In various embodiments, the antibiotic resistance gene is a β-lactamase, vancomycin and/or macrolide resistance gene. In various embodiments, the antibiotic resistance gene is a CfxA family of β-lactamases or VanRD/VanSD gene. In various embodiments, the antibiotic resistance gene is one or more of acrE, acrF, acrS, AmpC, baeR, cfxA, cpxR, ermY, marA, mdtD, mdtN, mdtK, pbp2, pbp4, and VanRD/VanSD.

In various embodiments, the antibiotic for which there is resistance, or for which the beta-lactamase helps induce a therapeutic response is different antibiotic is a selected from beta-lactams, carbapenems, monobactams, β-lactamase inhibitors, aminoglycosides, tetracyclines, rifamycins, macrolides, ketolides, lincosamides, streptogramins, sulphonamides, oxazolidinones, and quinolones.

In various embodiments, the present methods prevent or reduce the onset of resistance to various antibiotics.

For instance, such antibiotics include β-lactam antibiotics, including penicillins and cephalosporins.

### DESCRIPTION OF THE FIGURES

FIG. 1 shows a schematic timeline of the pig study of the Examples.

FIG. 2A shows a heat map analysis of the frequency of all antibiotic-resistance genes in the pig fecal microbiomes. FIG. 2B shows a heat map analysis of the frequency of beta-lactamase genes in the pig fecal microbiomes.

FIG. 3 shows changes in the frequency of selected antibiotic resistance genes. The data is from the study of Example 1.

FIG. 4A and FIG. 4B show changes in the frequency of selected antibiotic resistance genes. The data is from the study of Example 1.

FIG. 5A to FIG. 5D show changes in the frequency of selected antibiotic resistance genes. The data is from the study of Example 2 (i.e. with amoxicillin or ertapenem treatment).

FIG. 6 shows amoxicillin serum levels in dogs treated with amoxicillin alone (black) or amoxicillin+SYN-004 (white). The data are displayed as area under the curve (mean and standard deviation). Day 1 data are displayed on the left panel, and Day 6 data are displayed on the right panel. Data were compared using one-way ANOVA with Dunnett's multiple comparison tests and p values were 0.703 for day 1 and 0.098 for day 6 data.

FIG. 7 shows a principal coordinate analysis. Three component principal coordinate analysis of fecal microbiomes. Fecal microbiomes from each animal were analyzed via principal coordinate analysis using the Bray-Curtis distance measure. Amoxicillin alone pretreatment (yellow), post treatment (green). Amoxicillin+SYN-004 pretreatment (pink) and post treatment (brown). The two circles on the right include only green dots.

FIG. 8 shows changes in the frequency of selected beta-lactamase genes. The relative frequency of the selected antibiotic resistance genes TEM-113 (left) and OXA-136 (right), are displayed from amoxicillin alone (left panels) or amoxicillin+SYN-004 (right panels). Each data point represents the relative gene frequency in each animal's microbiome from pretreatment to post treatment.

FIG. 9 shows changes in the frequency of selected antibiotic resistance genes. The change in the relative frequency (mean) of the indicated antibiotic resistance genes for the amoxicillin alone (black bars) or amoxicillin+ribaxamase (white bars) from pretreatment compared to post treatment. A negative value indicates a reduction in the frequency, a positive value indicates an increased frequency. The genes are listed on the horizontal axis.

### DETAILED DESCRIPTION

In various aspects, the present invention relates to a method for treating or preventing an infection in a patient determined to be resistant to an antibiotic by administering an effective amount of a beta-lactamase having an amino acid sequence of at least 95% (or 97%, or 98%, or 99%, or 100%) identity with SEQ ID NO: 1 (and optionally having an Ambler D276N mutation) before or concurrently with the antibiotic or a different antibiotic.

In various embodiments, the patient is resistant to the antibiotic and the beta-lactamase provides a therapeutic response to the same antibiotic or the patient is resistant to the antibiotic and the beta-lactamase provides a therapeutic response to the different antibiotic.

In various embodiments, resistance to an antibiotic is detected using an antimicrobial susceptibility test (AST) or one or more sequencing methods (e.g., by determining by detecting the presence, absence, or level of one or more genes associated with resistance in a biological sample (e.g., stool or an aspirate of GI tract fluid) from the patient.

In various embodiments, the antibiotic resistance gene is one of the genes listed in Table A. In various embodiments, the antibiotic resistance gene is a β-lactamase, vancomycin and/or macrolide resistance gene. In various embodiments, the antibiotic resistance gene is a CfxA family of β-lactamases or VanRD/VanSD gene. In various embodiments, the antibiotic resistance gene is one or more of acrE, acrF, acrS, AmpC, baeR, cfxA, cpxR, ermY, marA, mdtD, mdtN, mdtK, pbp2, pbp4, and VanRD/VanSD.

In various embodiments, the antibiotic for which there is resistance, or for which the beta-lactamase helps induce a therapeutic response is different antibiotic is a selected from beta-lactams, carbapenems, monobactams, β-lactamase inhibitors, aminoglycosides, tetracyclines, rifamycins, macrolides, ketolides, lincosamides, streptogramins, sulphonamides, oxazolidinones, and quinolones.

Methods of Treatment

In various embodiments, the present invention relates to methods of antibiotic administration to a subject without promoting, or by preventing or mitigating, antibiotic resistance. In various embodiments, the present invention relates to the use of therapeutic beta-lactamases to modulate the microbiome, including the gastrointestinal (GI) microbiome, to reduce or prevent resistance to antibiotics. In various embodiments, the present invention relates to the use of therapeutic beta-lactamases to preserve a resistome state of a subject before administration of an antibiotic, e.g., to reduce or prevent an increased resistome presence. In various embodiments, the present invention relates to the use of therapeutic beta-lactamases to reduce a resistome state of a subject despite administration of an antibiotic.

Accordingly, in various embodiments, the present invention allows for increased antibiotic options for a subject. For instance, a subject may harbor microbes having resistance to any of the antibiotics described herein and accordingly be unlikely to respond to treatment with such antibiotic. Administration of the present beta-lactamase increases the likelihood of antibiotic response, even for antibiotics for which resistance is expected, by, without wishing to be bound by theory, modulating the GI microbiome to disfavor resistance.

In various embodiments, the present invention relates to methods and compositions for preventing the emergence of antibiotic resistance with therapeutic beta-lactamases. In various embodiments, the present invention relates to methods and compositions for preventing the emergence of antibiotic resistant organisms in the gut microbiome.

In various embodiments, the present invention relates to methods and compositions for reducing or eliminating antibiotic resistance with therapeutic beta-lactamases. In various embodiments, the present invention relates to methods and compositions for reducing or eliminating antibiotic resistant organisms in the gut microbiome with therapeutic beta-lactamases.

In various embodiments, the present methods allow for maintenance of, or reduction of, a subject's resistome despite selective antibiotic pressure being applied by antibiotic administration. Accordingly, in various embodiments, the present methods reduce or prevent the emergence of opportunistic pathogens that carry resistance.

In various embodiments, the patient is resistant to one or more antibiotics. For instance, a patient being resistant may be determined using an antimicrobial susceptibility test (AST) or one or more sequencing methods (e.g., by determining by detecting the presence, absence, or level of one or more genes associated with resistance in a biological sample (e.g., stool or an aspirate of GI tract fluid) from the patient. Further, a patient being resistant may be indicated by a prior failure of treatment with one or more antibiotics.

In various embodiments, the present invention allows for the screening of one or more antibiotic resistance genes, e.g., as described herein, to assess treatment options, e.g., selection of an antibiotic. For example, in some embodiments, the subject is evaluated for a state of an antibiotic resistance gene to determine if an antibiotic should be administered and/or if the present beta-lactamase should be administered to supplement an antibiotic. By way of example, the present invention provides, in some embodiments, screening a subject for one or more antibiotic resistance genes before an antibiotic treatment and, if there is an increased expression indicative of resistance, co-administering the present beta-lactamase to reduce or eliminate such resistance. Alternatively, in some embodiments, the present invention provides screening a subject for one or more antibiotic resistance genes early in the course of an antibiotic treatment and if an increased expression indicative of resistance is observed, administering the present beta-lactamase to reduce or eliminate such resistance.

In various embodiments, the present methods result in a quantifiable change in resistance or expression of resistance genes, e.g., of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the resistance.

In various embodiments, the present methods result in a quantifiable change in resistance or expression of resistance genes, e.g., of at least about 2-fold, or 5-fold, or 10-fold, or 15-fold, or 20-fold, or 25-fold, or 50-fold. In some embodiments, the present methods result in a reduction in resistance or expression of resistance genes, e.g., of at least about 2-fold, or 5-fold, or 10-fold, or 15-fold, or 20-fold, or 25-fold, or 50-fold relative to a control sample (e.g., not being resistance to an antibiotic).

In various embodiments, the antibiotic resistance gene is a beta-lactamase gene, such as blaOXA, encoding extended spectrum OXA class D beta-lactamases, blaCTX-M_82, blaCFX_A4, encoding extended spectrum class A serine beta-lactamases, and AmpC, encoding the extended spectrum cephalosporin-resistant class C beta-lactamases; a multidrug efflux transporter system gene such as acrE, encoding a component of the AcrEF-ToIC multidrug efflux transporter system (Lau and Zgurskaya, 2005, J. Bacteriol. 187:7815); baeR; encoding a response regulator of the MdtABC multidrug efflux transporter system (Nagakubo et al., 2002, J. Bacteriol. 184:4161); emrY, encoding a component of the EmrKY-ToIC multidrug efflux transporter system (Tanabe et al., 1997, J. Gen. Appl. Microbiol. 43:257); mdtD, encoding a component of the MdtABC multidrug efflux transporter system (Nagakubo et al., 2002, J. Bacteriol. 184:4161); and mdtN, encoding a multidrug resistance efflux pump from the major facilitator superfamily (Sulavik et al., 2001, Antimicrob. Agents Chemother. 45:1126); pbp2, encoding penicillin binding protein 2 (Bharat et al., 2015, Antimicrob. Agents Chemother. 59:5003); pbp4, encoding penicillin binding protein 4 (Sun et al., 2014, PLoS One 9:e97202); andaminoglycoside_strA (Scholz et al., 1989, Gene 75:271) encodes an aminoglycoside phosphotransferase, and Tetracycline_tet39 (Agerso and Guardabassi, 2005, J. Antimicrob. Chemother. 55:566) encodes a component of a tetracycline efflux pump.

Other antibiotic resistance genes are provided in the Antibiotic Resistance Genes Database (ARDB), see Nucl. Acids Res. (2009) 37 (suppl 1): D443-D447, the World Wide Web (www) at ardb.cbcb.umd.edu, Antimicrob. Agents Chemother. July 2013 vol. 57 no. 7 3348-3357, and the NCBI database (the World Wide Web (www) at ncbi.nlm.nih.gov), the entire contents of which are hereby incorporated by reference.

In various embodiments, the antibiotic resistance gene is one or more of the genes listed in Table A:

Aminocoumarins:
Aminocoumarin-resistant DNA topoisomerases
Aminocoumarin-resistant GyrB, ParE, ParY
Aminoglycosides:
Aminoglycoside acetyltransferases
AAC(1), AAC(2′), AAC(3), AAC(6′)
Aminoglycoside nucleotidyltransferases
ANT(2″), ANT(3″), ANT(4′), ANT(6), ANT(9)
Aminoglycoside phosphotransferases
APH(2″), APH(3″), APH(3′), APH(4), APH(6), APH(7″), APH(9)
16S rRNA methyltransferases
ArmA, RmtA, RmtB, RmtC, Sgm
β-Lactams:
Class A β-lactamases
AER, BLA1, CTX-M, KPC, SHV, TEM, etc.
Class B (metallo-)β-lactamases
BlaB, CcrA, IMP, NDM, VIM, etc.
Class C β-lactamases
ACT, AmpC, CMY, LAT, PDC, etc.
Class D β-lactamases
OXA β-lactamase
mecA (methicillin-resistant PBP2)
Mutant porin proteins conferring antibiotic resistance
Antibiotic-resistant Omp36, OmpF, PIB (por)
Genes modulating β-lactam resistance:
bla (blal, blaR1) and mec (mecl, mecR1) operons
Chloramphenicol:
Chloramphenicol acetyltransferase (CAT)
Chloramphenicol phosphotransferase
Ethambutol:
Ethambutol-resistant arabinosyltransferase (EmbB)
Mupirocin:
Mupirocin-resistant isoleucyl-tRNA synthetases
MupA, MupB
Peptide antibiotics:
Integral membrane protein MprF
Phenicol:
Cfr 23S rRNA methyltransferase
Rifampin:
Rifampin ADP-ribosyltransferase (Arr)
Rifampin glycosyltransferase
Rifampin monooxygenase
Rifampin phosphotransferase
Rifampin resistance RNA polymerase-binding proteins
DnaA, RbpA
Rifampin-resistant beta-subunit of RNA polymerase (RpoB)
Streptogramins:
Cfr 23S rRNA methyltransferase
Erm 23S rRNA methyltransferases
ErmA, ErmB, Erm(31), etc.
Streptogramin resistance ATP-binding cassette (ABC) efflux pumps
Lsa, MsrA, Vga, VgaB
Streptogramin Vgb lyase
Vat acetyltransferase
Fluoroquinolones:
Fluoroquinolone acetyltransferase
Fluoroquinolone-resistant DNA topoisomerases
Fluoroquinolone-resistant GyrA, GyrB, ParC
Quinolone resistance protein (Qnr)
Fosfomycin:
Fosfomycin phosphotransferases
FomA, FomB, FosC
Fosfomycin thiol transferases
FosA, FosB, FosX
Glycopeptides:
VanA, VanB, VanD, VanR, VanS, etc.
Lincosamides:
Cfr 23S rRNA methyltransferase
Erm 23S rRNA methyltransferases
ErmA, ErmB, Erm(31), etc.
Lincosamide nucleotidyltransferase (Lin)
Linezolid:
Cfr 23S rRNA methyltransferase
Macrolides:
Cfr 23S rRNA methyltransferase
Erm 23S rRNA methyltransferases
ErmA, ErmB, Erm(31), etc.
Macrolide esterases
EreA, EreB
Macrolide glycosyltransferases
GimA, Mgt, Ole
Macrolide phosphotransferases (MPH)
MPH(2′)-I, MPH(2′)-II
Macrolide resistance efflux pumps
MefA, MefE, Mel
Streptothricin:
Streptothricin acetyltransferase (sat)
Sulfonamides:
Sulfonamide-resistant dihydropteroate synthases
Sul1, Sul2, Sul3, sulfonamide-resistant FolP
Tetracyclines:
Mutant porin PIB (por) with reduced permeability
Tetracycline inactivation enzyme TetX
Tetracycline resistance major facilitator superfamily (MFS) efflux pumps
TetA, TetB, TetC, Tet30, Tet31, etc.
Tetracycline resistance ribosomal protection proteins
TetM, TetO, TetQ, Tet32, Tet36, etc.
Efflux pumps conferring antibiotic resistance:
ABC antibiotic efflux pump
MacAB-TolC, MsbA, MsrA, VgaB, etc.
MFS antibiotic efflux pump
EmrD, EmrAB-TolC, NorB, GepA, etc.
Multidrug and toxic compound extrusion (MATE) transporter
MepA
Resistance-nodulation-cell division (RND) efflux pump
AdeABC, AcrD, MexAB-OprM, mtrCDE, etc.
Small multidrug resistance (SMR) antibiotic efflux pump
EmrE
Genes modulating antibiotic efflux:
adeR, acrR, baeSR, mexR, phoPQ, mtrR, etc.

In various embodiments, the resistance genes are tetracycline resistant genes. In various embodiments, the resistance genes are genes involved in β-lactam (including cephalosporin) resistance (bla) like TEM-1 and CMY. More than 300 β-lactam resistance genes have been identified and these are categorized by Ambler classes A to D and are possible genes of the present invention. Briefly, classes A, C and D are serine proteases while class B are Zn²⁺ metallo proteases like the recently described NDM-1. These enzymes all degrade β-lactam antibiotics by hydrolyzing the β-lactam ring. There are also non-enzymatic mechanisms of β-lactam resistance like the modified penicillin binding protein 2 (PBP2a) of the methicillin resistant Staphylococcus aureus, and other organisms also use similar mechanisms of resistance.

Class A beta-lactamases (Ambler classification) refer to serine beta-lactamases, in which hydrolysis of beta-lactam is mediated by serine in the active site, usually at amino acid position 70 in the alpha helix₂. Class A beta-lactamases include but are not limited to Len-1, SHV-1, TEM-1, PSE-3/PSE-3, ROB-1, Bacillus cereus such as 5/B type 1, 569/H type 1 and 569/H type 3, Bacillus anthrasis spp., Bacillus licheniformis such as PenP, Bacillus weihenstephanensis, Bacillus clausii, Staphylococcus aureus, PC1, Sme-1, NmcA, IMI-, PER-, VEB-, GES-, KPC-, CME- and CTX-M types beta-lactamases.

In some embodiments, the resistance gene is beta-lactamase of class EC 3.5.2.6, e.g., selected from a functional Group 1, Group 2, Group 3, or a Group 4 beta-lactamase (see, e.g., Bush et al., Antimicrob. Agents Chemother, 39: 1211, the contents of which are hereby incorporated by reference). Without wishing to be bound by theory, Group 1 consists of cephalosporinases that are not well inhibited by clavulanic acid; Group 2 consists of penicillinases, cephalosporinases and broad-spectrum beta-lactamases that are generally inhibited by active site-directed beta-lactamase inhibitors; Group 3 consists of metallo-beta-lactamases that hydrolyze penicillins, cephalosporins and carbapenems, and that are poorly inhibited by almost all beta-lactam-containing molecules; and Group 4 consists of penicillinases that are not well inhibited by clavulanic acid) and/or a molecular/Ambler class A, or class B, or class C, or class D beta-lactamase (see, e.g., Ambler 1980, Philos Trans R Soc Lond B Biol Sci. 289: 321 the contents of which are hereby incorporated by reference), without wishing to be bound by theory: Classes A, C, and D gather evolutionarily distinct groups of serine beta-lactamase enzymes, and class B the zinc-dependent (“EDTA-inhibited”) beta-lactamase enzymes (see Ambler R. P. et al., 1991, Biochem J. 276: 269-270, the contents of which are hereby incorporated by reference). In some embodiments, the antibiotic degradation enzyme is a serine beta-lactamase or a zinc-dependent (EDTA-inhibited) beta-lactamase. For example, in some embodiments, the beta-lactamase is an extended-spectrum beta-lactamase (ESBL), optionally selected from a TEM, SHV, CTX-M, OXA, PER, VEB, GES, and IBC beta-lactamase. Further, the beta-lactamase may be an inhibitor-resistant β-lactamase, optionally selected from an AmpC-type β-lactamases, Carbapenemase, IMP-type carbapenemases (metallo-β-lactamases), VIM (Verona integron-encoded metallo-β-lactamase), OXA (oxacillinase) group of β-lactamases, KPC (K. pneumonia carbapenemase), CMY (Class C), SME, IMI, NMC and CcrA, and a NDM (New Delhi metallo-β-lactamase, e.g., NDM-1) beta-lactamase.

In various embodiments, the present methods relate to the reduction or removal of resistance genes associated with ceftriaxone use, such as one or more of:

| Gene | Function | Found in, for example |
|---|---|---|
| blaCMY-2 | AmpC type β-lactamase | Salmonella |
| blaTEM-1 | Ambler class A β-lactamase | Salmonella |
| blaVIM-1 | Zn²⁺ metallo | Enterobacteriaceae and others |
| blaNDM-1 | Zn²⁺ metallo protease | Klebsiella and other |
| | | Enterobacteriaceae |
| floR | phenicol-specific efflux pump | Salmonella enterica |
| blaSHV-18 | Ambler class A β-lactamase | K. pneumoniae and E. coli |

In various embodiments, the antibiotic resistance gene is a β-lactamase, vancomycin and/or macrolide resistance gene.

In various embodiments, the antibiotic resistance gene is a CfxA family of β-lactamases or VanRD/VanSD gene.

In various embodiments, the antibiotic resistance gene is one or more of acrE, acrF, acrS, AmpC, baeR, cfxA, cpxR, ermY, marA, mdtD, mdtN, mdtK, pbp2, pbp4, and VanRD/VanSD.

In various embodiments, the present methods allow for screening or assessment of antibiotic resistance, e.g., by determining the presence, absence or level of one or more antibiotic-resistance genes. Illustrative suitable samples include fecal samples (e.g., stool), as well as aspirates of the fluid in the GI tract, mucosal biopsies from a site in the gastrointestinal tract, and other tissue samples or tissue homogenates.

Illustrative methods of antibiotic resistance gene detection are known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in, for example, Innis, et al. (1990) PCR Protocols, A Guide to Methods and Applications, Academic Press, Inc. N.Y.). Measurement of DNA copy number at microsatellite loci using quantitative PCR analysis is described in, for example, Ginzonger, et al. (2000) Cancer Research 60:5405-5409.

The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and Sybr green.

Other suitable amplification methods include, but are not limited to, ligase chain reaction (LCR) (see, for example, Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

In still other embodiments of the methods provided herein, sequencing of individual nucleic molecules (or their amplification products) is performed. In one embodiment, a high throughput parallel sequencing technique that isolates single nucleic acid molecules of a population of nucleic acid molecules prior to sequencing may be used. Such strategies may use so-called “next generation sequencing systems” including, without limitation, sequencing machines and/or strategies well known in the art, such as those developed by Illumina/Solexa (the Genome Analyzer; Bennett et al. (2005) Pharmacogenomics, 6:373-20 382), by Applied Biosystems, Inc. (the SOLiD Sequencer; solid.appliedbiosystems.com), by Roche (e.g., the 454 GS FLX sequencer; Margulies et al. (2005) Nature, 437:376-380; U.S. Pat. Nos. 6,274,320; 6,258,568; 6,210,891) and others. Other sequencing strategies such as stochastic sequencing (e.g., as developed by Oxford Nanopore) may also be used, e.g., as described in International Patent Publication No. WO 2010/004273.

In still other embodiments of the methods provided herein, deep sequencing can be used to identify and quantify a resistant microorganism. These techniques are known in the art.

In various embodiments, 16S rRNA sequencing is used. In various embodiments, fecal DNA samples are analyzed by whole genome shotgun sequencing (WGS), which can resolve individual antibiotic resistance genes within a sample.

Metagenomic, whole genome shotgun sequencing (WGS) enables an analysis of the near-complete genomic content of the collection of microbes in a particular sample, also referred to as the pangenome (depth of sequencing directly relates to the amount of the pangenome that is covered). Illustrative methods that can be used were developed for the NIH-Human Microbiome Project. Briefly, bacterial genomic DNA, available from 16S rRNA sequencing work, is expected to be used for WGS. Individual libraries constructed from each sample are loaded onto the HiSeq platform (Illumina) and sequenced using the 2×100 bp pair-end read protocol. Illumina paired-end libraries are constructed from total genomic DNA isolated from each sample. The DNA is sheared into approximately 400-600 bp fragments followed by ligation of Illumina adaptors containing molecular barcodes for downstream de-multiplexing. These products are then amplified through ligation-mediated PCR (LM-PCR) using, e.g., KAPA HiFi DNA Polymerase (Kapa Biosystems, Wilmington, MA, USA). Following bead purification, with e.g., Agencourt AMPure XP (Beckman Coulter, Brea, CA, USA), quantification and size distribution of the LM-PCR product is determined using, e.g., the LabChip GX electrophoresis system (PerkinElmer, Akron, OH, USA). Libraries are be pooled in equimolar amounts at six samples per pool, and prepared for sequencing with TruSeq PE Cluster Generation Kit (Illumina). Each library pool is loaded onto one lane of a HiSeq 2000 flow cell spiked with 1% PhiX control library. Sequencing files are de-multiplexed with CASAVA version 1.8.3 (Illumina). Quality filtering, trimming and de-multiplexing is carried out by a custom pipeline containing Trim Galore and cutadapt for adaptor and quality trimming, and PRINSEQ for low complexity filtering and sequence deduplication. In addition, Bowtie2 v2.2.1 will be used to map reads to MetaPhlAn markers for the classification of bacterial species. There are several databases that are available to map results for the identification of antibiotic resistant genes in the isolated DNA samples from the fecal microbiome, these include the comprehensive antibiotic resistance database (CARD), as well as the antibiotic resistance genes database (ARDB, the World Wide Web (www) at ardb.cbcb.umd.edu) and the NCBI database (the World Wide Web (www) at ncbi.nlm.nih.gov).

Further, in various embodiments, quantitative polymerase chain reaction (qPCR) is used to detect increases in the number of certain antibiotic resistance genes. The available literature can be consulted for publicly available primers for the antibiotic resistance genes of or new primers can be designed, e.g., using Primer Express Software (Applied Biosystems) and the genomic information available at the time of access. Quantitative PCR sample analysis can be performed in a QuantStudio 7 Real-Time PCR System, using MicroAmp Fast Optical 96-well reaction plates (0.1 ml) and MicroAmp Optical 384-Well Reaction Plates, MicroAmp optical adhesive film (all Applied Biosystems), and PerfeCta SYBR Green FastMix, Low Rox PCR Master Mix (Quanta Biosciences).

In order to normalize the qPCR reactions across the various samples, qPCR of the 16S rRNA in the samples can be conducted and then the ratio of antibiotic resistance genes to 16S rRNA can be normalized.

In various embodiments, the present methods find use in the treatment or prevention of infections by one or more of the following pathogens: Aeromonas hydrophila, Bacillus, e.g., Bacillus cereus, Bifidobacterium, Bordetella, Borrelia, Brucella, Burkholderia, C. difficile, Campylobacter, e.g., Campylobacter fetus and Campylobacter jejuni, Chlamydia, Chlamydophila, Clostridium, e.g., Clostridium botulinum, Clostridium difficile, and Clostridium perfringens, Corynebacterium, Coxiella, Ehrlichia, Enterobacteriaceae, e.g., Carbapenem-resistant Enterobacteriaceae (CRE) and Extended Spectrum Beta-Lactamase producing Enterobacteriaceae (ESBL-E), fluoroquinolone-resistant Enterobacteriaceae, Enterococcus, e.g., vancomycin-resistant enterococcus spp., extended spectrum beta-lactam resistant Enterococci (ESBL), and vancomycin-resistant Enterococci (VRE), Escherichia, e.g., enteroaggregative Escherichia coli, enterohemorrhagic Escherichia coli, enteroinvasive Escherichia coli, enteropathogenic E. coli, enterotoxigenic Escherichia coli (such as but not limited to LT and/or ST), Escherichia coli 0157:H7, and multi-drug resistant bacteria Escherichia coli, Francisella, Haemophilus, Helicobacter, e.g., Helicobacter pylon, Klebsiella, e.g., Klebsiella pneumonia and multi-drug resistant bacteria Klebsiella, Legionella, Leptospira, Listeria, e.g., Lysteria monocytogenes, Morganella, Mycobacterium, Mycoplasma, Neisseria, Orientia, Plesiomonas shigelloides, Antibiotic-resistant Proteobacteria, Proteus, Pseudomonas, Rickettsia, Salmonella, e.g., Salmonella paratyphi, Salmonella spp., and Salmonella typhi, Shigella, e.g., Shigella spp., Staphylococcus, e.g., Staphylococcus aureus and Staphylococcus spp., Streptococcus, Treponema, Vibrio, e.g., Vibrio cholerae, Vibrio parahaemolyticus, Vibrio spp., and Vibrio vulnificus, and Yersinia, e.g., Yersinia enterocolitica.

In various embodiments, the present methods find use in the treatment or prevention of infection an antibiotic-resistant bacterium, e.g., Antibiotic-resistant Proteobacteria, Vancomycin Resistant Enterococcus (VRE), Carbapenem Resistant Enterobacteriaceae (CRE), fluoroquinolone-resistant Enterobacteriaceae, andr Extended Spectrum Beta-Lactamase producing Enterobacteriaceae (ESBL-E).

In various embodiments, the patient in need thereof can be in an outpatient setting, hospitalized and/or in a long-term care facility. In various embodiments, a subject in need thereof has or is at risk for a bloodstream infection (BSI), catheter or intravascular-line infections (e.g., central-line infections), chronic inflammatory diseases, meningitis, pneumonia, e.g., ventilator-associated pneumonia, skin and soft tissue infections, surgical-site infections, urinary tract infections (e.g., antibiotic-resistant urinary tract infections and catheter-associated urinary tract infections), wound infections, and/or other well-known infections: antibiotic-resistant infections and antibiotic-sensitive infections.

In various embodiments, the present methods find use in the treatment or prevention of C. difficile infection (CDI) and/or a C. difficile-associated disease.

In various embodiments, the present methods find use in the treatment or prevention of overgrowth of pathogenic bacteria such as vancomycin resistant enterococci (VRE).

In various embodiments, the present methods find use allowing treatment of the patient with one or more of ceftriaxone, cefotaxime, cefazolin, cefoperazone, cefuroxime, piperacillin, and vancomycin. In various embodiments, the present patient is resistant to one or more of ceftriaxone, cefotaxime, cefazolin, cefoperazone, cefuroxime, piperacillin, and vancomycin and the present beta-lactamases alter the patient's microbiome to restore therapeutic efficacy of one or more of ceftriaxone, cefotaxime, cefazolin, cefoperazone, cefuroxime, piperacillin, and vancomycin in the patient.

Beta-Lactamases

In some aspects, the present invention involves uses of one or more beta-lactamases, e.g., to reduce or prevent antibiotic resistance. As used herein, a beta-lactamase refers to an enzyme, which hydrolyzes beta-lactams. Hydrolysis of the amide bond of the beta-lactam ring makes the antimicrobial agents biologically inactive. As used herein, class A beta-lactamases (Ambler classification) refer to serine beta-lactamases, in which hydrolysis of beta-lactam is mediated by serine in the active site, usually at amino acid position 70 in the alpha helix₂. Class A beta-lactamases include but are not limited to Len-1, SHV-1, TEM-1, PSE-3/PSE-3, ROB-1, Bacillus cereus such as 5/B type 1, 569/H type 1 and 569/H type 3, Bacillus anthrasis sp, Bacillus licheniformis such as PenP, Bacillus weihenstephanensis, Bacillus clausii, Staphylococcus aureus, PC1, Sme-1, NmcA, IMI-, PER-, VEB-, GES-, KPC-, CME- and CTX-M types beta-lactamases.

In various aspects, the beta-lactamases has the amino acid sequence of SEQ ID NO: 1 (i.e., “SYN-004” or “ribaxamase” or “P3A” as described in WO 2011/148041, the entire contents of which are hereby incorporated by reference). Mutations may be made to this sequence to generate beta-lactamase derivatives that may be utilized by methods of the invention.

SEQ ID NO: 1
TEMKDDFAKLEEQFDAKLGIFALDTGTNRTVAYRPDERFAFASTIKALTV
GVLLQQKSIEDLNQRITYTRDDLVNYNPITEKHVDTGMTLKELADASLRY
SDNAAQNLILKQIGGPESLKKELRKIGDEVTNPERFEPELNEVNPGETQD
TSTARALVTSLRAFALEDKLPSEKRELLIDWMKRNTTGDALIRAGVPDGW
EVADKTGAASYGTRNDIAIIWPPKGDPVVLAVLSSRDKKDAKYDNKLIAE
ATKVVMKALNMNGK.

In some embodiments, the beta-lactamase comprises an amino acid sequence having at least about 60% (e.g., about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 1.

In some embodiments, SEQ ID NO: 1 may have a Met and/or Thr preceding the first residue of the sequence. In various embodiments, the Met may be cleaved. As described herein, mutations may be made to the sequence comprising the Met and/or Thr preceding the first residue to generate beta-lactamase derivatives. In some embodiments, the leading Thr may bring about increased stability of the enzyme relative to another leading amino acid (e.g., Lys). For example, such a residue may confer increased resistance to an amino peptidase.

Also provided herein is the nucleotide sequence of SYN-004 as SEQ ID NO: 2:

SEQ ID NO: 2
atgactgagatgaaagatgattttgcgaagctggaagaacagtttgacgc
aaaattgggcattttcgcgttggacacgggtacgaatcgtacggttgcct
accgtccggacgagcgcttcgccttcgcgagcacgatcaaagccctgacc
gtcggcgtgctgctccagcaaaagagcatcgaggacctgaaccagcgcat
tacctacacccgtgatgatctggtgaactataatccgatcaccgagaaac
acgttgataccggtatgaccctgaaagaactggcagatgcaagcctgcgc
tacagcgataacgcggctcagaatctgattctgaagcaaatcggtggtcc
ggagagcttgaagaaagaactgcgtaaaatcggcgatgaagtcactaatc
cggagcgttttgagccggagctgaacgaagtgaatccgggtgaaacgcaa
gacacgagcaccgcgcgtgcgcttgtcacctccctgcgcgctttcgcact
ggaagataagctgccgtcggagaaacgcgagctgctgatcgactggatga
agcgcaatacgaccggcgacgcgctgattcgtgcgggcgttccggacggt
tgggaagtggctgacaagaccggtgcggcgagctacggcacccgtaacga
tatcgcgatcatttggccacctaaaggtgacccggtcgtgctggccgtac
tgagcagccgtgacaagaaagacgcaaagtatgataacaagctgattgca
gaggcgaccaaagttgttatgaaggcactgaacatgaatggtaag

In some embodiments, a polynucleotide of the invention may have at least about 60% (e.g., about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 2.

In some embodiments, the beta-lactamase, e.g., SYN-004, has substantial ceftriaxone hydrolyzing activity. In some embodiments, the beta-lactamase, e.g., SYN-004, hydrolyzes ceftriaxone substantially more efficiently than P1A.

In illustrative embodiments, the beta-lactamases comprise an amino acid sequence having at least 60% sequence identity with SEQ ID NO: 1 and the following of Ambler classification: a hydrophobic residue other than alanine (A) at position 232; a hydrophilic residue other than alanine (A) at position 237; a hydrophobic residue other than alanine (A) at position 238; a hydrophilic residue other than serine (S) at position 240; and a hydrophilic residue other than aspartate (D) at position 276. In some embodiments, the hydrophobic residue other than alanine (A) at position 232 is glycine (G). In some embodiments, the hydrophilic residue other than alanine (A) at position 237 is serine (S). In some embodiments, the hydrophobic residue other than alanine (A) at position 238 is glycine (G). In some embodiments, the hydrophilic residue other than serine (S) at position 240 is aspartate (D). In some embodiments, the other than aspartate (D) at position 276 is asparagine (N). In some embodiments, the beta-lactamase comprises one or more of A232G, A237S, A238G, S240D, and D276N. In some embodiments, the beta-lactamase comprises all of A232G, A237S, A238G, S240D, and D276N, the sequence of which is SEQ ID NO: 3, i.e. P4A. In some embodiments, the beta-lactamase and/or pharmaceutical composition comprises an amino acid sequence having at least 90%, or 95%, or 97%, or 99%, or 100% sequence identity with SEQ ID NO: 3.

SEQ ID NO: 3
EMKDDFAKLEEQFDAKLGIFALDTGTNRTVAYRPDERFAFASTIKALTVG
VLLQQKSIEDLNQRITTRDDLVNYNPITEKHVDTGMTLKELADASLRYSD
NAAQNLILKQIGGPESLKKELRKIGDEVTNPERFEPELNEVNPGETQDTS
TARALVTSLRAFALEDKLPSEKRELLIDWMKRNTTGDALIRAGVPDGWEV
GDKTGSGDYGTRNDIAIIWPPKGDPVVLAVLSSRDKKDAKYDNKLIAEAT
KVVMKALNMNGK

In some embodiments, the beta-lactamase polypeptide of the invention comprises an amino acid sequence having at least about 60% (e.g., about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 3.

SEQ ID NO: 4, is derived from SEQ ID NO: 3, and further includes the signal and the addition of the QASKT amino acids (the coding region is underlined):

MIQKRKRTVSFRLVLMCTLLFVSLPITKTSAQASKTEMKDDFAKLEEQFD
AKLGIFALDTGTNRTVAYRPDERFAFASTIKALTVGVLLQQKSIEDLNQR
ITYTRDDLVNYNPITEKHVDTGMTLKELADASLRYSDNAAQNLILKQIGG
PESLKKELRKIGDEVTNPERFEPELNEVNPGETQDTSTARALVTSLRAFA
LEDKLPSEKRELLIDWMKRNTTGDALIRAGVPDGWEVGDKTGSGDYGTRN
DIAIIWPPKGDPVVLAVLSSRDKKDAKYDNKLIAEATKVMKALNMNGK

In some embodiments, the beta-lactamase polypeptide of the invention comprises an amino acid sequence having at least about 60% (e.g., about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 4.

In some embodiments, the beta-lactamase and/or pharmaceutical composition comprises an amino acid sequence having at least 90%, or 95%, or 97%, or 99%, or 100% sequence identity with SEQ ID NO: 4.

An illustrative polynucleotide of the invention is SEQ ID NO: 5, which is the full nucleotide sequence of A232G, A237S, A238G, S240D, and D276N mutant, Hind III site (AAGCTT-in bold) and additional K and T amino acids. In some embodiments, the underlined portion of SEQ ID NO: 5, is omitted. The leader and additional nucleotides (Hind III site and K and T amino acids—for the addition of the amino acid sequence QASKT) are underlined.

atgattcaaaaacgaaagcggacagtttcgttcagacttgtgcttatgtg
cacgctgttatttgtcagtttgccgattacaaaaacatcagcgcaagctt
ccaagacggagatgaaagatgattttgcaaaacttgaggaacaatttgat
gcaaaactcgggatctttgcattggatacaggtacaaaccggacggtagc
gtatcggccggatgagcgttttgcttttgcttcgacgattaaggctttaa
ctgtaggcgtgcttttgcaacagaaatcaatagaagatctgaaccagaga
ataacatatacacgtgatgatcttgtaaactacaacccgattacggaaaa
gcacgttgatacgggaatgacgctcaaagagcttgcggatgcttcgcttc
gatatagtgacaatgcggcacagaatctcattcttaaacaaattggcgga
cctgaaagtttgaaaaaggaactgaggaagattggtgatgaggttacaaa
tcccgaacgattcgaaccagagttaaatgaagtgaatccgggtgaaactc
aggataccagtacagcaagagcacttgtcacaagccttcgagcctttgct
cttgaagataaacttccaagtgaaaaacgcgagcttttaatcgattggat
gaaacgaaataccactggagacgccttaatccgtgccggtgtgccggacg
gttgggaagtgggtgataaaactggaagcggagattatggaacccggaat
gacattgccatcatttggccgccaaaaggagatcctgtcgttcttgcagt
attatccagcagggataaaaaggacgccaagtatgataataaacttattg
cagaggcaacaaaggtggtaatgaaagccttaaacatgaacggcaaataa

In some embodiments, the polynucleotide of the present invention has at least about 60% (e.g., about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 5 (with or without the underlined portion).

In various aspects, the beta-lactamase polypeptide has the sequence of SEQ ID NO: 6 (i.e., P2A) or is derived by one or more mutations of SEQ ID NO: 6:

ETGTISISQLNKNVWVHTELGYENGEAVPSNGLVLNTSKGLVLVDSSWDN
KLTKELIEMVEKKFQKRVTDVIITHAHADRIGGITALKERGIKAHSTALT
AELAKNSGYEEPLGDLQTITSLKFGNTKVETFYPGKGHTEDNIVVWLPQY
QILAGGCLVKSAEAKDLGNVADAYVNEWSTSIENVLKRYGNINSVVPGHG
EVGDKGLLLHTLDLLK.

In some embodiments, the beta-lactamase polypeptide of the invention comprises an amino acid sequence having at least about 60% (e.g., about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with SEQ ID NO: 6.

In some embodiments, the beta-lactamase and/or pharmaceutical composition comprises an amino acid sequence having at least 90%, or 95%, or 97%, or 99%, or 100% sequence identity with SEQ ID NO: 6.

Additional sequences of beta-lactamases including P1A (i.e. SEQ ID NO: 1 except position 276 is D and not N), P2A, P3A/SYN-004, and P4A and derivatives thereof are described for example, in WO 2011/148041 and PCT/US2015/026457, the entire contents of which are hereby incorporated by reference.

Further, the beta-lactamase polypeptide may include additional upstream residues from the first residue of SEQ ID NO: 1 (see, e.g., JBC 258 (18): 11211, 1983, the contents of which are hereby incorporated by reference-including the exo-large and exo-small versions of penP and penP1). Further, the beta-lactamase polypeptide may also include additional downstream residues from the last residue of SEQ ID NO: 1.

In some embodiments, the beta-lactamase includes one or more (e.g., about 1, or about 2, or about 3, or about 4, or about 5, or about 6, or about 7, or about 8, or about 9, or about 10) mutations relative to SEQ ID NO: 1 or SEQ ID NO: 2. In some embodiments the beta-lactamase includes a variant of SYN-004, e.g., a sequence with at least 95, 96, 97, 98, 99, 99.5, 99.8, 99.9% identity to SEQ ID NO: 1 or SEQ ID NO: 2. In various embodiments, one or more amino acid of SEQ ID NO: 1 is substituted with a naturally occurring amino acid, such as a hydrophilic amino acid (e.g., a polar and positively charged hydrophilic amino acid, such as arginine (R) or lysine (K); a polar and neutral of charge hydrophilic amino acid, such as asparagine (N), glutamine (Q), serine (S), threonine (T), proline (P), and cysteine (C), a polar and negatively charged hydrophilic amino acid, such as aspartate (D) or glutamate (E), or an aromatic, polar and positively charged hydrophilic amino acid, such as histidine (H)) or a hydrophobic amino acid (e.g., a hydrophobic, aliphatic amino acid such as glycine (G), alanine (A), leucine (L), isoleucine (I), methionine (M), or valine (V), a hydrophobic, aromatic amino acid, such as phenylalanine (F), tryptophan (W), or tyrosine (Y) or a non-classical amino acid (e.g., selenocysteine, pyrrolysine, N-formylmethionine β-alanine, GABA and δ-Aminolevulinic acid. 4-Aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general). In some embodiments, SEQ ID NO: 1 may have a Met and/or Thr preceding the first residue of the sequence. These residues may be similarly mutated as above.

Illustrative mutants include:

| Mutations relative to P1A (based on | |
|---|---|
| the Ambler classification) | Name |
| Wild type | RS310 (or P1A) |
| D276N | IS118 (or SYN-004) |
| I72S | IS222 |
| T160F | IS203 |
| R244T | IS217 |
| R244T D276K | IS215 |
| Q135M | IS197 |
| G156R A238T | IS235 |
| F33Y D276N | IS158 |
| F33Y S240P D276N | IS230 (or IS181) |
| F33Y A238T D276N | IS232 (or IS180) |
| I72S Q135M T160F (Block 1 mutants) | IS227 |
| A232G A237S A238G S240D (Block 2 | IS191 |
| mutants) | |
| A232G A237S A238G S240D R244T | IS229 |
| A232G A237S A238G S240D D276R | IS219 |
| A232G A237S A238G S240D D276K | IS221 |
| A232G A237S A238G S240D Q135M | IS224 |
| A238T | IS233 |
| T243I S266N D276N | IS234 (or IS176) |
| A232G A237S A238G S240D D276N | IS288 (or P4A) |

In all of these mutants, the numbering of residues corresponds to SEQ ID NO: 1. These residue numbers may be converted to Ambler numbers (Ambler et al., 1991, A standard numbering scheme for the Class A β-lactamases, Biochem. J. 276:269-272, the contents of which are hereby incorporated by reference) through use of any conventional bioinformatic method, for example by using BLAST (Basic Local Alignment Search Tools) or FASTA (FAST-All).

In various embodiments, the beta-lactamase used in the invention is produced in bacterial cells such as an E. coli cell (see, e.g., PCT/US15/47187, the entire contents of which are hereby incorporated by reference).

Formulations/Modified Release Profile

In various embodiments, the present invention employs modified release formulations comprising at least one beta-lactamase, wherein the formulation releases a substantial amount of the beta-lactamase into one or more regions of the GI tract. In some embodiments, the beta-lactamase is SYN-004, or the other beta-lactamase agents described herein, and variants thereof (e.g., as described above). For example, the formulation may release at least about 60% of the beta-lactamase, for example, SYN-004, after the stomach and into one or more regions of the GI tract.

In various embodiments, the modified-release formulations of the present invention are designed for immediate release (e.g., upon ingestion). In various embodiments, the modified-release formulations may have sustained-release profiles, i.e. slow release of the active ingredient(s) in the body (e.g., GI tract) over an extended period of time. In various embodiments, the modified-release formulations may have a delayed-release profile, i.e. not immediately release the active ingredient(s) upon ingestion; rather, postponement of the release of the active ingredient(s) until the composition is lower in the gastrointestinal tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric coated to delay release of the active ingredient(s) until it reaches the small intestine or large intestine. In some embodiments, there is not a substantial amount of the active ingredient(s) of the present formulations in the stool.

In various embodiments, the modified-release formulation of the present invention releases at least 60% of the beta-lactamase (e.g., SYN-004, or the other beta-lactamase agents described herein, and variants thereof) after the stomach into one or more regions of the intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the beta-lactamase (e.g., SYN-004, or the other beta-lactamase agents described herein, and variants thereof) in the intestine.

In various embodiments, the modified-release formulation of the present invention releases at least 60% of the beta-lactamase (e.g., SYN-004, or the other beta-lactamase agents described herein, and variants thereof) in the small intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the beta-lactamase (e.g., SYN-004, or the other beta-lactamase agents described herein, and variants thereof) in the small intestine.

In one embodiment, the modified-release formulation of the present invention releases at least 60% of the beta-lactamase (e.g., SYN-004, or the other beta-lactamase agents described herein, and variants thereof) in the duodenum. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the beta-lactamase (e.g., SYN-004, or the other beta-lactamase agents described herein, and variants thereof) in the duodenum.

In one embodiment, the modified-release formulation of the present invention releases at least 60% of the beta-lactamase (e.g., SYN-004, or the other beta-lactamase agents described herein, and variants thereof) in the jejunum. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the beta-lactamase (e.g., SYN-004, or the other beta-lactamase agents described herein, and variants thereof) in the jejunum.

In one embodiment, the modified-release formulation of the present invention releases at least 60% of the beta-lactamase (e.g., SYN-004, or the other beta-lactamase agents described herein, and variants thereof) in the ileum and/or the ileocecal junction. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the beta-lactamase (e.g., SYN-004, or the other beta-lactamase agents described herein, and variants thereof) in the ileum and/or the ileocecal junction.

In various embodiments, the modified-release formulation of the present invention releases at least 60% of the beta-lactamase (e.g., SYN-004, or the other beta-lactamase agents described herein, and variants thereof) in the large intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the beta-lactamase (e.g., SYN-004, or the other beta-lactamase agents described herein, and variants thereof) in the large intestine.

In one embodiment, the modified-release formulation of the present invention releases at least 60% of the beta-lactamase (e.g., SYN-004, or the other beta-lactamase agents described herein, and variants thereof) in the cecum. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the beta-lactamase (e.g., SYN-004, or the other beta-lactamase agents described herein, and variants thereof) in the cecum.

In one embodiment, the modified-release formulation of the present invention releases at least 60% of the beta-lactamase (e.g., SYN-004, or the other beta-lactamase agents described herein, and variants thereof) in the ascending colon. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the beta-lactamase (e.g., SYN-004, or the other beta-lactamase agents described herein, and variants thereof) in the ascending colon.

In one embodiment, the modified-release formulation of the present invention releases at least 60% of the beta-lactamase (e.g., SYN-004, or the other beta-lactamase agents described herein, and variants thereof) in the transverse colon. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the beta-lactamase (e.g., SYN-004, or the other beta-lactamase agents described herein, and variants thereof) in the transverse colon.

In one embodiment, the modified-release formulation of the present invention releases at least 60% of the beta-lactamase (e.g., SYN-004, or the other beta-lactamase agents described herein, and variants thereof) in the descending colon. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the beta-lactamase (e.g., SYN-004, or the other beta-lactamase agents described herein, and variants thereof) in the descending colon.

In one embodiment, the modified-release formulation of the present invention releases at least 60% of the beta-lactamase (e.g., SYN-004, or the other beta-lactamase agents described herein, and variants thereof) in the sigmoid colon. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the beta-lactamase (e.g., SYN-004, or the other beta-lactamase agents described herein, and variants thereof) in the sigmoid colon.

In various embodiments, the modified-release formulation does not substantially release the beta-lactamase (e.g., SYN-004, or the other beta-lactamase agents described herein, and variants thereof) in the stomach.

In certain embodiments, the modified-release formulation releases the beta-lactamase (e.g., SYN-004, or the other beta-lactamase agents described herein, and variants thereof) at a specific pH. For example, in some embodiments, the modified-release formulation is substantially stable in an acidic environment and substantially unstable (e.g., dissolves rapidly or is physically unstable) in a near neutral to alkaline environment. In some embodiments, stability is indicative of not substantially releasing while instability is indicative of substantially releasing. For example, in some embodiments, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some embodiments, the present formulations are stable in lower pH areas and therefore do not substantially release in, for example, the stomach. In some embodiments, modified-release formulation is substantially stable at a pH of about 1 to about 4 or lower and substantially unstable at pH values that are greater. In these embodiments, the modified-release formulation is not substantially released in the stomach. In these embodiments, the modified-release formulation is substantially released in the small intestine (e.g., one or more of the duodenum, jejunum, and ileum) and/or large intestine (e.g., one or more of the cecum, ascending colon, transverse colon, descending colon, and sigmoid colon). In some embodiments, modified-release formulation is substantially stable at a pH of about 4 to about 5 or lower and consequentially is substantially unstable at pH values that are greater and therefore is not substantially released in the stomach and/or small intestine (e.g., one or more of the duodenum, jejunum, and ileum). In these embodiments, the modified-release formulation is substantially released in the large intestine (e.g., one or more of the cecum, ascending colon, transverse colon, descending colon, and sigmoid colon). In various embodiments, the pH values recited herein may be adjusted as known in the art to account for the state of the subject, e.g., whether in a fasting or postprandial state.

In some embodiments, the modified-release formulation is substantially stable in gastric fluid and substantially unstable in intestinal fluid and, accordingly, is substantially released in the small intestine (e.g., one or more of the duodenum, jejunum, and ileum) and/or large intestine (e.g., one or more of the cecum, ascending colon, transverse colon, descending colon, and sigmoid colon).

In some embodiments, the modified-release formulation is stable in gastric fluid or stable in acidic environments. These modified-release formulations release about 30% or less by weight of the beta-lactamase (e.g., SYN-004, or the other beta-lactamase agents described herein, and variants thereof) and/or additional therapeutic agent in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of the of the invention may release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the beta-lactamase (e.g., SYN-004, or the other beta-lactamase agents described herein, and variants thereof) and/or additional therapeutic agent in the modified-release formulation in gastric fluid with a pH of 4-5, or less or simulated gastric fluid with a pH of 4-5 or less, in about 15, or about 30, or about 45, or about 60, or about 90 minutes. Modified-release formulations of the invention may release about 1%, about 2%, about 3%, about 4%, about 5%, about 6%, about 7%, about 8%, about 9%, or about 10% by weight of the total beta-lactamase (e.g., SYN-004, or the other beta-lactamase agents described herein, and variants thereof) and/or additional therapeutic agent in the modified-release formulation in gastric fluid with a pH of 5 or less, or simulated gastric fluid with a pH of 5 or less, in about 15, or about 30, or about 45, or about 60, or about 90 minutes.

In some embodiments, the modified-release formulation is unstable in intestinal fluid. These modified-release formulations release about 70% or more by weight of the beta-lactamase (e.g., SYN-004, or the other beta-lactamase agents described herein, and variants thereof) and/or additional therapeutic agent in the modified-release formulation in intestinal fluid or simulated intestinal fluid in about 15, or about 30, or about 45, or about 60, or about 90 minutes. In some embodiments, the modified-release formulation is unstable in near neutral to alkaline environments. These modified-release formulations release about 70% or more by weight of the beta-lactamase (e.g., SYN-004, or the other beta-lactamase agents described herein, and variants thereof) and/or additional therapeutic agent in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments may release 70% or more by weight of beta-lactamase (e.g., SYN-004, or the other beta-lactamase agents described herein, and variants thereof) and/or additional therapeutic agent in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In one embodiment, the modified-release formulation may remain essentially intact, or may be essentially insoluble, in gastric fluid. The modified-release formulation may include one or more delayed-release coatings that are pH dependent. Delayed-release coatings that are pH dependent will be substantially stable in acidic environments (pH of about 5 or less), and substantially unstable in near neutral to alkaline environments (pH greater than about 5). For example, the delayed-release coating may essentially disintegrate or dissolve in near neutral to alkaline environments such as are found in the small intestine (e.g., one or more of the duodenum, jejunum, and ileum) and/or large intestine (e.g., one or more of the cecum, ascending colon, transverse colon, descending colon, and sigmoid colon).

Alternatively, the stability of the modified-release formulation can be enzyme-dependent. In such embodiments, the modified-release formulation may include one or more delayed-release coatings that are enzyme-dependent.

Delayed-release coating that are enzyme-dependent will be substantially stable in fluid that does not contain a particular enzyme and substantially unstable in fluid containing the enzyme. The delayed-release coating will essentially disintegrate or dissolve in fluid containing the appropriate enzyme. Enzyme-dependent control can be brought about, for example, by using materials which release the active ingredient only on exposure to enzymes in the intestine, such as galactomannans. Also, the stability of the modified-release formulation can be dependent on enzyme stability in the presence of a microbial enzyme present in the gut flora.

In various embodiments, the modified-release formulations comprising a beta-lactamase (e.g., SYN-004, or variants thereof) are substantially stable in chyme. For example, there is, in some embodiments, a loss of less about 50% or about 40%, or about 30%, or about 20%, or about 10% of beta-lactamase activity in about 10, or 9, or 8, or 7, or 6, or 5, or 4, or 3, or 2, or 1 hour from administration.

In some embodiments, a dual pulse formulation is provided. In various embodiments, the present invention provides for modified-release formulations that release multiple doses of the beta-lactamase (e.g., SYN-004, or the other beta-lactamase agents described herein, and variants thereof), at different locations along the intestines, at different times, and/or at different pH. In an illustrative embodiment, the modified-release formulation comprises a first dose of the beta-lactamase and a second dose of the beta-lactamase, wherein the first dose and the second dose are released at different locations along the intestines, at different times, and/or at different pH. For example, the first dose is released at the duodenum, and the second dose is released at the ileum. In another example, the first dose is released at the jejunum, and the second dose is released at the ileum. In other embodiments, the first dose is released at a location along the small intestine (e.g., the duodenum), while the second dose is released along the large intestine (e.g., the ascending colon). In various embodiments, the modified-release formulation may release at least one dose, at least two doses, at least three doses, at least four doses, at least five doses, at least six doses, at least seven doses, or at least eight doses of the beta-lactamase (e.g., SYN-004, or the other beta-lactamase agents described herein, and variants thereof) at different locations along the intestines, at different times, and/or at different pH. Further the dual pulse description herein applies to modified-release formulations that release a beta-lactamase (e.g., SYN-004, or the other beta-lactamase agents described herein, and variants thereof) and an additional therapeutic agent.

In various embodiments, the present invention uses a modified-release formulation of beta-lactamase (e.g., SYN-004, or the other beta-lactamase agents described herein, and variants thereof) which may further comprise a pharmaceutically acceptable carrier or excipient. As one skilled in the art will recognize, the formulations can be in any suitable form appropriate for the desired use and route of administration.

In some embodiments, the administration of the modified-release formulation including beta-lactamase (and/or additional therapeutic agents) is any one of oral, intravenous, and parenteral. In some embodiments, the administration of the modified-release formulation including beta-lactamase (and/or additional agents) is not intravenous in order to, for example, prevent interference with an antibiotic administered systemically. In other embodiments, routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically, particularly to the ears, nose, eyes, or skin.

Any modified-release formulation including beta-lactamase (and/or additional therapeutic agents) as described herein can be administered orally. Such inventive formulations can also be administered by any other convenient route, for example, by intravenous infusion or bolus injection, by absorption through epithelial or mucocutaneous linings (e.g., oral mucosa, rectal and intestinal mucosa, etc.) and can be administered together with an additional therapeutic agent. Administration can be systemic or local. In some embodiments, administration is not at the site of infection to avoid, for example, hydrolysis of an antibiotic at the site of infection. Various delivery systems are known, e.g., encapsulation in liposomes, microparticles, microcapsules, capsules, etc., and can be used for administration. In specific embodiments, it may be desirable to administer locally to the area in need of treatment.

Suitable dosage forms for oral use include, for example, solid dosage forms such as tablets, dispersible powders, granules, and capsules. In one embodiment, the modified-release formulation is in the form of a capsule. In another embodiment, the modified-release formulation is in the form of a tablet. In yet another embodiment, the modified-release formulation is in the form of a soft-gel capsule. In a further embodiment, the modified-release formulation is in the form of a gelatin or hydroxypropyl methylcellulose (HPMC) capsule.

In some dosage forms, the agents described herein are mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients may have two or more functions in the oral dosage form. In the case of an oral dosage form, for example, a capsule or a tablet, the dosage form may also comprise buffering agents.

The modified release formulation can additionally include a surface active agent. Surface active agents suitable for use in the present invention include, but are not limited to, any pharmaceutically acceptable, non-toxic surfactant. Classes of surfactants suitable for use in the compositions of the invention include, but are not limited to polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some embodiments, compositions of the invention may comprise one or more surfactants including, but not limited to, sodium lauryl sulfate, polysorbate 20, polysorbate 40, polysorbate 60, polysorbate 80, and triethyl citrate.

The modified-release formulation can also contain pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

The modified-release formulation can also include one or more application solvents. Some of the more common solvents that can be used to apply, for example, a delayed-release coating composition include isopropyl alcohol, acetone, methylene chloride and the like.

The modified-release formulation can also include one or more alkaline materials. Alkaline material suitable for use in compositions of the invention include, but are not limited to, sodium, potassium, calcium, magnesium and aluminum salts of acids such as phosphoric acid, carbonic acid, citric acid and other aluminum/magnesium compounds. In addition the alkaline material may be selected from antacid materials such as aluminum hydroxides, calcium hydroxides, magnesium hydroxides and magnesium oxide.

The solid oral dosage forms can be prepared by, for example granulation (e.g., wet or dry granulation) of the agents of the invention with one or more suitable excipients. Alternatively, the agents of the invention can be layered onto an inert core (e.g., a nonpareil/sugar sphere such as a sucrose sphere or silica sphere) using conventional methods such as fluidized bed or pan coating, or extruded and spheronized using methods known in the art, into active compound-containing pellets. In embodiment, the beta-lactamase (e.g., SYN-004, or the other beta-lactamase agents described herein, and variants thereof) is spray-coated onto a sucrose sphere.

Such pellets can then be incorporated into tablets or capsules using conventional methods.

Suspensions, in addition to the active agents, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Besides inert diluents, the oral compositions can also include adjuvants such as sweetening, flavoring, and perfuming agents.

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The formulations comprising the beta-lactamase (and/or additional therapeutic agents) may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, the modified-release formulation of the present invention may utilize one or more modified-release coatings such as delayed-release coatings to provide for effective, delayed yet substantial delivery of the beta-lactamase (e.g., SYN-004, or the other beta-lactamase agents described herein, and variants thereof) to the GI tract together with, optionally, other additional therapeutic agents.

In one embodiment, the delayed-release coating includes an enteric agent that is substantially stable in acidic environments and substantially unstable in near neutral to alkaline environments. In an embodiment, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymers include, for example, EUDRAGIT® FS 30D, L 30 D-55, L 100-55, L 100, L 12,5, L 12,5 P, RL 30 D, RL PO, RL 100, RL 12,5, RS 30 D, RS PO, RS 100, RS 12,5, NE 30 D, NE 40 D, NM 30 D, S 100, S 12,5, and S 12,5 P. Similar polymers include Kollicoat® MAE 30 DP and Kollicoat® MAE 100 P. In some embodiments, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 S 12,5 P, Kollicoat® MAE 30 DP and Kollicoat® MAE 100 P is used. In various embodiments, the enteric agent may be a combination of the foregoing solutions or dispersions. In an embodiment, the delayed-release coating includes the enteric agent EUDRAGIT® L 30 D-55.

In certain embodiments, one or more coating system additives are used with the enteric agent. For example, one or more PIasACRYL™ additives may be used as an anti-tacking agent coating additive. Exemplary PIasACRYL™ additives include, but are not limited to PIasACRYL™ HTP20 and PIasACRYL™ T20. In an embodiment, PIasACRYL™ HTP20 is formulated with EUDRAGIT® L 30 D-55 coatings. In another embodiment, PIasACRYL™ T20 is formulated with EUDRAGIT® FS 30 D coatings.

In another embodiment, the delayed-release coating may degrade as a function of time when in aqueous solution without regard to the pH and/or presence of enzymes in the solution. Such a coating may comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings may be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers useful in the present invention include polyvinyl esters, polyvinyl acetals, polyacrylic acid esters, butadiene styrene copolymers, and the like. In one embodiment, colonic delivery is achieved by use of a slowly-eroding wax plug (e.g., various PEGS, including for example, PEG6000).

In a further embodiment, the delayed-release coating may be degraded by a microbial enzyme present in the gut flora. In one embodiment, the delayed-release coating may be degraded by a bacteria present in the small intestine. In another embodiment, the delayed-release coating may be degraded by a bacteria present in the large intestine.

In various embodiments, the invention provides a formulation comprising: a core particle having a base coat comprising one or more beta-lactamases (e.g., SYN-004, or the other beta-lactamase agents described herein, and variants thereof), and a delayed-release coating disposed over the coated core particle. The delayed-release coating may be substantially stable in acidic environments and/or gastric fluid, and/or substantially unstable in near neutral to alkaline environments or intestinal fluid thereby exposing the coated core particle to intestinal fluid. The base coat comprising one or more beta-lactamases may further comprise one or more additional therapeutic agents. Optionally a plurality of base coats may be applied to the core each of which may contain a beta-lactamase and/or an additional therapeutic agent. In an embodiment, the core particle includes sucrose. The formulation can be prepared by methods known in the art. For example, a beta-lactamases (e.g., SYN-004, or the other beta-lactamase agents described herein, and variants thereof) can be sprayed onto an inert core (e.g., a sucrose core or sucrose sphere) and spray-dried with an enteric layer (e.g., EUDRAGIT L30 D-55) to form beta-lactamase (e.g., SYN-004, or the other beta-lactamase agents described herein, and variants thereof)-containing pellets.

Optionally, the core particle may comprise one or more beta-lactamases (e.g., SYN-004, or the other beta-lactamase agents described herein, and variants thereof) and/or one or more additional therapeutic agents. In one embodiment, one or more doses of the beta-lactamase may be encapsulated in a core particle, for example, in the form of a microsphere. For example, the beta-lactamase may be combined with a polymer (e.g., latex), and then formed into a particulate, micro-encapsulated enzyme preparation, without using a sucrose core. The microspheres thus formed may be optionally covered with a delayed-release coating.

A variety of approaches for generating particulates (such as microspheres, aggregates, other) are known which are amenable to the inclusion of enzymes. They typically involve at least two phases, one containing the enzyme, and one containing a polymer that forms the backbone of the particulate. Most common are coacervation, where the polymer is made to separate from its solvent phase by addition of a third component, or multiple phase emulsions, such as water in oil in water (w/o/w) emulsion where the inner water phase contains the protein, the intermediate organic phase contains the polymer, and the external water phase stabilizers that support the w/o/w double emulsion until the solvents can be removed to form the microspheres. Alternatively, the beta-lactamase (e.g., SYN-004, or the other beta-lactamase agents described herein, and variants thereof) and stabilizing excipients (for example, trehalose, mannitol, Tween 80, polyvinyl alcohol) are combined and sprayed from aqueous solution and collected. The particles are then suspended in a dry, water immiscible organic solvent containing polymer and release modifying compounds, and the suspension sonicated to disperse the particles. An additional approach uses aqueous phases but no organic solvent. Specifically, the enzyme, buffer components, a polymer latex, and stabilizing and release-modifying excipients are dissolved/dispersed in water. The aqueous dispersion is spray-dried, leading to coalescence of the latex, and incorporation of the protein and excipients in particles of the coalesced latex. When the release modifiers are insoluble at acidic conditions but soluble at higher pHs (such as carboxylic acid) then release from the matrix is inhibited in the gastric environment.

In some embodiments, before applying the delayed-release coating to the coated core particle the particle can optionally be covered with one or more separating layers comprising pharmaceutical excipients including alkaline compounds such as for instance pH-buffering compounds. The separating layer essentially separates the coated core particle from the delayed-release coating.

The separating layer can be applied to the coated core particle by coating or layering procedures typically used with coating equipment such as a coating pan, coating granulator or in a fluidized bed apparatus using water and/or organic solvents for the coating process. As an alternative the separating layer can be applied to the core material by using a powder coating technique. The materials for separating layers are pharmaceutically acceptable compounds such as, for instance, sugar, polyethylene glycol, polyvinylpyrrolidone, polyvinyl alcohol, polyvinyl acetate, hydroxypropyl cellulose, methyl-cellulose, ethylcellulose, hydroxypropyl methylcellulose, carboxymethylcellulose sodium and others, used alone or in mixtures. Additives such as plasticizers, colorants, pigments, fillers, anti-tacking and anti-static agents, such as for instance magnesium stearate, titanium dioxide, talc and other additives can also be included in the separating layer.

In some embodiments, the coated particles with the delayed-release coating may be further covered with an overcoat layer. The overcoat layer can be applied as described for the other coating compositions. The overcoat materials are pharmaceutically acceptable compounds such as sugar, polyethylene glycol, polyvinylpyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures. The overcoat materials can prevent potential agglomeration of particles coated with the delayed-release coating, protect the delayed-release coating from cracking during the compaction process or enhance the tableting process.

In various embodiments, the formulation may comprise a plurality of modified-release particles or pellets or microspheres. In one embodiment, the formulation is in the form of capsules comprising multiple pellets. In one embodiment, the formulation is in the form of capsules comprising multiple microspheres.

In some embodiments, the modified-release formulation is a capsule filled with a plurality of beta-lactamase-containing pellets (e.g., SYN-004 (or the other beta-lactamase agents described herein, and variants thereof)-containing pellets) from which the beta-lactamase is released. In an embodiment, the capsule is a gelatin capsule, such as a hard gelatin capsule. In another embodiment, the capsule is a hydroxypropyl methylcellulose (HPMC) capsule. For example, the formulation may be in the form of capsules comprising multiple pellets. For example, the formulation may be in the form of capsules such as, for example, gelatin or hydroxypropyl methylcellulose (HPMC) capsules comprising multiple enteric-coated pellets containing beta-lactamase (e.g., SYN-004, or the other beta-lactamase agents described herein, and variants thereof). In such an embodiment, a combination of pellets may be utilized in which each pellet is designed to release at a specific time point or location. In various embodiments, the pellets (e.g., enteric-coated pellets) are designed to pass through the stomach unchanged and then release the beta-lactamase (e.g., SYN-004, or the other beta-lactamase agents described herein, and variants thereof) into one or more regions of the intestines. In some embodiments, the beta-lactamase-containing pellets may be enteric-coated to release the beta-lactamase (e.g., SYN-004, or the other beta-lactamase agents described herein, and variants thereof) at different intestinal pH values.

In various embodiments, the formulation of the present invention is in the form of a capsule (e.g., a hard gelatin or HPMC capsule) comprising a plurality of enteric-coated beta-lactamase-containing pellets. In such embodiments, the pellets (or each individual pellet) comprise a beta-lactamase (e.g., SYN-004, or the other beta-lactamase agents described herein, and variants thereof), a sucrose sphere, which the beta-lactamase, for example, SYN-004 or a variant, is sprayed onto, a binder excipient (e.g., hydroxypropylcellulose (HPC)), an enteric polymer (e.g., EUDRAGIT L 30 D-55), a plasticizer (e.g., triethyl citrate), a glidant (e.g., glyceryl monostearate), an emulsifier, and buffer salts.

In various embodiments, the formulation of the present invention is in the form of a capsule (e.g., a hard gelatin or HPMC capsule) comprising a plurality of enteric-coated beta-lactamase-containing pellets. In such embodiments, the pellets (or each individual pellet) comprise about 10-20% by weight of beta-lactamase (e.g., SYN-004, or the other beta-lactamase agents described herein, and variants thereof). For example, the beta-lactamase (e.g., SYN-004, or the other beta-lactamase agents described herein, and variants thereof) may be present at about 10%, about 11%, about 12%, about 13%, about 14%, about 15%, about 16%, about 17%, about 18%, about 19%, or about 20% by weight. In some embodiments, the pellets (or each individual pellet) comprise about 20-30% by weight sucrose sphere, which the beta-lactamase, for example, SYN-004 or a variant, is sprayed onto. For example, the sucrose sphere may be present at about 20%, about 21%, about 22%, about 23%, about 24%, about 25%, about 26%, about 27%, about 28%, about 29%, or about 30% by weight. In various embodiments, the pellets (or each individual pellet) comprise about 30-40% by weight a binder excipient (e.g., hydroxypropylcellulose (HPC)). For example, the binder excipient may be present at about 30%, about 31%, about 32%, about 33%, about 34%, about 35%, about 36%, about 37%, about 38%, about 39%, or about 40% by weight. In some embodiments, the pellets (or each individual pellet) comprise about 15-25% by weight an enteric polymer (e.g., EUDRAGIT L 30 D-55). For example, the enteric polymer may be present at about 15%, about 16%, about 17%, about 18%, about 19%, about 20%, about 21%, about 22%, about 23%, about 24%, or about 25% by weight. In some embodiments, the pellets (or each individual pellet) comprise about 1.5-2.5% by weight of plasticizer (e.g., triethyl citrate). For example, the plasticizer may be present at about 1.5%, about 1.6%, about 1.7%, about 1.8%, about 1.9%, about 2%, about 2.1%, about 2.2%, about 2.3%, about 2.4%, about 2.5% by weight. In some embodiments, the pellets (or each individual pellet) comprise about 0.5-1.5% by weight glidant (e.g., glyceryl monostearate). For example, the glidant may be present at about 0.5%, about 0.6%, about 0.7%, about 0.8%, about 0.9%, about 1%, about 1.1%, about 1.2%, about 1.3%, about 1.4%, or about 1.5% by weight. In some embodiments, the pellets (or each individual pellet) comprise about 0.1-1.0% by weight emulsifier (e.g., polysorbate-80). For example, the emulsifier may be present at about 0.1%, about 0.2%, about 0.3%, about 0.4%, about 0.5%, about 0.6%, about 0.7%, about 0.8%, about 0.9%, or about 1% by weight. In some embodiments, the pellets (or each individual pellet) further comprise about 1-2% by weight buffer salts. For example, the buffer salts may be present at about 1.1%, about 1.2%, about 1.3%, about 1.4%, about 1.5%, about 1.6%, about 1.7%, about 1.8%, about 1.9%, or about 2% by weight. The weight as described herein refers to the total weight of all components excluding the weight of the capsule itself.

In some embodiments, the pellets (or each individual pellet) comprise about 16% by weight of the beta-lactamase (e.g., SYN-004, or the other beta-lactamase agents described herein, and variants thereof); about 23% by weight sucrose sphere; about 35% by weight a binder excipient (e.g., hydroxypropylcellulose (HPC)); about 21% by weight an enteric polymer (e.g., EUDRAGIT L 30 D-55); about 2% by weight of plasticizer (e.g., triethyl citrate); about 1% by weight glidant (e.g., glyceryl monostearate); about 0.5% by weight emulsifier (e.g., polysorbate-80); and about 2% by weight buffer salts. The weight as described herein refers to the total weight of all components excluding the weight of the capsule itself.

For example, the pellets (or each individual pellet) comprise about 15.8% by weight of the beta-lactamase (e.g., SYN-004, or the other beta-lactamase agents described herein, and variants thereof); about 23.3% by weight sucrose sphere; about 35% by weight a binder excipient (e.g., hydroxypropylcellulose (HPC)); about 20.8% by weight an enteric polymer (e.g., EUDRAGIT L 30 D-55); about 2.1% by weight of plasticizer (e.g., triethyl citrate); about 1.0% by weight glidant (e.g., glyceryl monostearate); about 0.4% by weight emulsifier (e.g., polysorbate-80); and about 1.6% by weight buffer salts. The weight as described herein refers to the total weight of all components excluding the weight of the capsule itself.

In various embodiments, the formulation of the present invention is in the form of a capsule (e.g., a hard gelatin or HPMC capsule) comprising about 75 mg of the beta-lactamase (e.g., SYN-004, or the other beta-lactamase agents described herein, and variants thereof). The capsule includes a plurality of enteric-coated beta-lactamase-containing pellets. In such embodiments, the formulation comprises about 10-20% by weight of the beta-lactamase (e.g., SYN-004, or the other beta-lactamase agents described herein, and variants thereof). For example, the beta-lactamase (e.g., SYN-004, or the other beta-lactamase agents described herein, and variants thereof) may be present at about 10%, about 11%, about 12%, about 13%, about 14%, about 15%, about 16%, about 17%, about 18%, about 19%, or about 20% by weight. In some embodiments, the formulation comprises about 15-25% by weight sucrose sphere. For example, the sucrose sphere may be present about 15%, about 16%, about 17%, about 18%, about 19%, about 20%, about 21%, about 22%, about 23%, about 24%, or about 25% by weight. In various embodiments, the formulation comprises about 25-35% by weight a binder excipient (e.g., hydroxypropylcellulose (HPC)). For example, the binder excipient may be present at about 25%, about 26%, about 27%, about 28%, about 29%, about 30%, about 31%, about 32%, about 33%, about 34%, or about 35% by weight. In some embodiments, the formulation comprises about 10-25% by weight an enteric polymer (e.g., EUDRAGIT L 30 D-55). For example, the enteric polymer may be present at about 10%, about 11%, about 12%, about 13%, about 14%, about 15%, about 16%, about 17%, about 18%, about 19%, about 20%, about 21%, about 22%, about 23%, about 24%, or about 25% by weight. In some embodiments, the formulation comprises about 1.5-2.5% by weight of plasticizer (e.g., triethyl citrate). For example, the plasticizer may be present at about 1.5%, about 1.6%, about 1.7%, about 1.8%, about 1.9%, about 2%, about 2.1%, about 2.2%, about 2.3%, about 2.4%, about 2.5% by weight. In some embodiments, the formulation comprises about 0.5-1.5% by weight glidant (e.g., glyceryl monostearate). For example, the glidant may be present at about 0.5%, about 0.6%, about 0.7%, about 0.8%, about 0.9%, about 1%, about 1.1%, about 1.2%, about 1.3%, about 1.4%, or about 1.5% by weight. In some embodiments, the formulation comprises about 0.1-1.0% by weight emulsifier (e.g., polysorbate-80). For example, the emulsifier may be present at about 0.1%, about 0.2%, about 0.3%, about 0.4%, about 0.5%, about 0.6%, about 0.7%, about 0.8%, about 0.9%, or about 1% by weight. In some embodiments, the formulation comprises about 1-2% by weight buffer salts. For example, the buffer salts may be present at about 1%, about 1.1%, about 1.2%, about 1.3%, about 1.4%, about 1.5%, about 1.6%, about 1.7%, about 1.8%, about 1.9%, or about 2% by weight. In some embodiments, the formulation comprises about 10-20% by weight gelatin or HPMC capsule. For example, the gelatin or HPMC capsule may be about 10%, about 11%, about 12%, about 13%, about 14%, about 15%, about 16%, about 17%, about 18%, about 19%, or about 20% by weight.

In some embodiments, the formulation of the present invention comprising about 75 mg of the beta-lactamase (e.g., SYN-004, or the other beta-lactamase agents described herein, and variants thereof). In such embodiments, the formulation comprises about 13% by weight of the beta-lactamase (e.g., SYN-004, or the other beta-lactamase agents described herein, and variants thereof); about 19% by weight sucrose sphere; about 29% by weight a binder excipient (e.g., hydroxypropylcellulose (HPC)); about 17% by weight an enteric polymer (e.g., EUDRAGIT L 30 D-55); about 2% by weight of plasticizer (e.g., triethyl citrate); about 1% by weight glidant (e.g., glyceryl monostearate); about 0.5% by weight emulsifier (e.g., polysorbate-80); about 1% by weight buffer salts; and about 17% by weight gelatin or HPMC capsule.

For example, the formulation comprises about 13.1% by weight of the beta-lactamase (e.g., SYN-004, or the other beta-lactamase agents described herein, and variants thereof); about 19.4% by weight sucrose sphere; about 29.1% by weight a binder excipient (e.g., hydroxypropylcellulose (HPC)); about 17.3% by weight an enteric polymer (e.g., EUDRAGIT L 30 D-55); about 1.7% by weight of plasticizer (e.g., triethyl citrate); about 0.9% by weight glidant (e.g., glyceryl monostearate); about 0.4% by weight emulsifier (e.g., polysorbate-80); about 1.3% by weight buffer salts; and about 16.8% by weight gelatin or HPMC capsule.

In various embodiments, the formulation of the present invention is in the form of a capsule (e.g., a hard gelatin or HPMC capsule) comprising about 25 mg of the beta-lactamase (e.g., SYN-004, or the other beta-lactamase agents described herein, and variants thereof). The capsule includes a plurality of enteric-coated beta-lactamase-containing pellets. In such embodiments, the formulation comprises about 5-15% by weight of the beta-lactamase (e.g., SYN-004, or the other beta-lactamase agents described herein, and variants thereof). For example, the beta-lactamase (e.g., SYN-004, or the other beta-lactamase agents described herein, and variants thereof) may be present at about 5%, about 6%, about 7%, about 8%, about 9%, about 10%, about 11%, about 12%, about 13%, about 14%, or about 15% by weight. In some embodiments, the formulation comprises about 10-20% by weight sucrose sphere. For example, the sucrose sphere may be present about 10%, about 11%, about 12%, about 13%, about 14%, about 15%, about 16%, about 17%, about 18%, about 19%, or about 20% by weight. In various embodiments, the formulation comprises about 15-25% by weight a binder excipient (e.g., hydroxypropylcellulose (HPC)). For example, the binder excipient may be present at about 15%, about 16%, about 17%, about 18%, about 19%, about 20%, about 21%, about 22%, about 23%, about 24%, or about 25% by weight. In some embodiments, the formulation comprises about 10-20% by weight an enteric polymer (e.g., EUDRAGIT L 30 D-55). For example, the enteric polymer may be present at about 10%, about 11%, about 12%, about 13%, about 14%, about 15%, about 16%, about 17%, about 18%, about 19%, or about 20% by weight. In some embodiments, the formulation comprises about 1.0-2.0% by weight of plasticizer (e.g., triethyl citrate).

For example, the plasticizer may be present at about 1.0%, about 1.1%, about 1.2%, about 1.3%, about 1.4%, about 1.5%, about 1.6%, about 1.7%, about 1.8%, about 1.9%, or about 2.0% by weight. In some embodiments, the formulation comprises about 0.1-1.0% by weight glidant (e.g., glyceryl monostearate). For example, the glidant may be present at about 0.1%, about 0.2%, about 0.3%, about 0.4%, about 0.5%, about 0.6%, about 0.7%, about 0.8%, about 0.9%, or about 1% by weight. In some embodiments, the formulation comprises about 0.1-1.0% by weight emulsifier (e.g., polysorbate-80). For example, the emulsifier may be present at about 0.1%, about 0.2%, about 0.3%, about 0.4%, about 0.5%, about 0.6%, about 0.7%, about 0.8%, about 0.9%, or about 1% by weight. In some embodiments, the formulation comprises about 0.5-1.5% by weight buffer salts. For example, the buffer salts may be present at about 0.5%, about 0.6%, about 0.7%, about 0.8%, about 0.9%, about 1.0%, about 1.1%, about 1.2%, about 1.3%, about 1.4%, or about 1.5% by weight. In some embodiments, the formulation comprises about 30-40% by weight gelatin or HPMC capsule. For example, the gelatin or HPMC capsule may be about 30%, about 31%, about 32%, about 33%, about 34%, about 35%, about 36%, about 37%, about 38%, about 39%, or about 40% by weight.

In some embodiments, the formulation of the present invention comprising about 25 mg of the beta-lactamase (e.g., SYN-004, or the other beta-lactamase agents described herein, and variants thereof). In such embodiments, the formulation comprises about 10% by weight of the beta-lactamase (e.g., SYN-004, or the other beta-lactamase agents described herein, and variants thereof); about 15% by weight sucrose sphere; about 22% by weight a binder excipient (e.g., hydroxypropylcellulose (HPC)); about 13% by weight an enteric polymer (e.g., EUDRAGIT L 30 D-55); about 1% by weight of plasticizer (e.g., triethyl citrate); about 0.5% by weight glidant (e.g., glyceryl monostearate); about 0.3% by weight emulsifier (e.g., polysorbate-80); about 1% by weight buffer salts; and about 38% by weight gelatin or HPMC capsule.

For example, the formulation comprises about 9.8% by weight of the beta-lactamase (e.g., SYN-004, or the other beta-lactamase agents described herein, and variants thereof); about 14.5% by weight sucrose sphere; about 21.8% by weight a binder excipient (e.g., hydroxypropylcellulose (HPC)); about 13% by weight an enteric polymer (e.g., EUDRAGIT L 30 D-55); about 1.3% by weight of plasticizer (e.g., triethyl citrate); about 0.6% by weight glidant (e.g., glyceryl monostearate); about 0.3% by weight emulsifier (e.g., polysorbate-80); about 1.0% by weight buffer salts; and about 37.7% by weight gelatin or HPMC capsule.

The present invention also provides for modified-release formulations that release multiple doses of the beta-lactamases (e.g., SYN-004, or the other beta-lactamase agents described herein, and variants thereof) and/or additional therapeutic agent along the gastrointestinal tract. In such embodiments, the overall release profile of such a formulation may be adjusted by utilizing, for example, multiple particle types or multiple layers. In one embodiment, the first dose of the beta-lactamase may be formulated for release in, for example, the small intestine (e.g., one or more of duodenum, jejunum, ileum) or the large intestine (e.g., one or more of cecum, ascending, transverse, descending or sigmoid portions of the colon, and rectum), whereas the second dose is formulated for delayed release in, for example, a different region of the small intestine (e.g., one or more of duodenum, jejunum, ileum) or the large intestine (e.g., one or more of cecum, ascending, transverse, descending or sigmoid portions of the colon, and rectum). Alternatively, multiple doses are released at different locations along the intestine. For example, in one embodiment, the first dose of the beta-lactamase may be formulated for release in, for example, the small intestine (e.g., one or more of duodenum, jejunum, ileum), whereas the second dose is formulated for delayed release in, for example, another part of the small intestine (e.g., one or more of duodenum, jejunum, ileum). In another embodiment, the first dose of the beta-lactamase may be formulated for release in, for example, the large intestine (e.g., one or more of cecum, ascending, transverse, descending or sigmoid portions of the colon, and rectum), whereas the second dose is formulated for delayed release in, for example, another part of the large intestine (e.g., one or more of cecum, ascending, transverse, descending or sigmoid portions of the colon, and rectum).

In various embodiments, the agents described herein may be in the form of a pharmaceutically acceptable salt, namely those salts which are suitable for use in contact with the tissues of humans and other animals without undue toxicity, irritation, allergic response and the like, and are commensurate with a reasonable benefit/risk ratio. Pharmaceutically acceptable salts are well known in the art. The salts can be prepared in situ during the final isolation and purification of the therapeutic agents, or separately by reacting the free base function with a suitable acid or a free acid functionality with an appropriate alkaline moiety. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

In various embodiments, the present formulations provide a number of advantages. For instance, the inventors have successfully formulated a protein (i.e. beta-lactamase), which itself is challenging. This is compounded further by the GI tract environment in which the present formulations release drug in various embodiments. Further, in various embodiments, the present formulations provide for GI tract release that is sufficiently slow to allow good protective coverage in the GI tract from adverse effects of various antibiotics, e.g., in the small intestine (a benefit that is accentuated by an increase in beta-lactamase half-life that is commensurate with a slower release). Furthermore, by coating the drug substance layer of the present pellets with HPC, as opposed to EUDRAGIT, for example, the present formulations minimize the amount of EUDRAGIT in the formulations and therefore mitigate possible dose-limiting toxicity and manufacturing complications.

Administration and Dosage

It will be appreciated that the actual dose of the beta-lactamase (e.g., SYN-004, or the other beta-lactamase agents described herein, and variants thereof) to be administered according to the present invention will vary according to, for example, the particular dosage form and the mode of administration. Many factors that may modify the action of the beta-lactamase (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

Individual doses of the beta-lactamase (e.g., SYN-004, or the other beta-lactamase agents described herein, and variants thereof) can be administered in unit dosage forms (e.g., tablets or capsules) containing, for example, from about 0.01 mg to about 1,000 mg, from about 0.01 mg to about 950 mg, from about 0.01 mg to about 900 mg, from about 0.01 mg to about 850 mg, from about 0.01 mg to about 800 mg, from about 0.01 mg to about 750 mg, from about 0.01 mg to about 700 mg, from about 0.01 mg to about 650 mg, from about 0.01 mg to about 600 mg, from about 0.01 mg to about 550 mg, from about 0.01 mg to about 500 mg, from about 0.01 mg to about 450 mg, from about 0.01 mg to about 400 mg, from about 0.01 mg to about 350 mg, from about 0.01 mg to about 300 mg, from about 0.01 mg to about 250 mg, from about 0.01 mg to about 200 mg, from about 0.01 mg to about 150 mg, from about 0.01 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg active ingredient, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg per unit dosage form, or from about 5 mg to about 80 mg per unit dosage form. For example, a unit dosage form can be about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, inclusive of all values and ranges therebetween. In an embodiment, individual dose of the beta-lactamase (e.g., SYN-004, or the other beta-lactamase agents described herein, and variants thereof) is administered in an unit dosage form containing 25 mg of the beta-lactamase. In another embodiment, individual dose of the beta-lactamase (e.g., SYN-004, or the other beta-lactamase agents described herein, and variants thereof) is administered in an unit dosage form containing 50 mg of the beta-lactamase. In a further embodiment, individual dose of the beta-lactamase (e.g., SYN-004, or the other beta-lactamase agents described herein, and variants thereof) is administered in an unit dosage form containing 75 mg of the beta-lactamase.

In one embodiment, the beta-lactamase is administered at an amount of from about 0.01 mg to about 100 mg daily, an amount of from about 0.01 mg to about 1,000 mg daily from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily.

In various embodiments, the beta-lactamase is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg, inclusive of all values and ranges therebetween.

In some embodiments, a suitable dosage of the beta-lactamase (e.g., SYN-004, or the other beta-lactamase agents described herein, and variants thereof) is in a range of about 0.01 mg/kg to about 100 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg body weight, inclusive of all values and ranges therebetween. In other embodiments, a suitable dosage of the beta-lactamases in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In various embodiments, the dose of SYN-004 is between about 75 mg to about 300 mg, e.g., about 75 mg, or about 100 mg, or about 125 mg, or about 150 mg, or about 175 mg, or about 200 mg, or about 225 mg, or about 250 mg, or about 275 mg, or about 300 mg.

In accordance with certain embodiments of the invention, the beta-lactamase may be administered, for example, about once per day, about every other day, about every third day, about once a week, about once every two weeks, about once every month, about once every two months, about once every three months, about once every six months, or about once every year. In certain embodiments, the beta-lactamase may be administered more than once daily, for example, about two times, about three times, about four times, about five times, about six times, about seven times, about eight times, about nine times, or about ten times daily.

Antibiotics

In various aspects, the present invention provides methods for preventing or reducing the generation of resistance to an antibiotic. In various embodiments, the subject is undergoing treatment or has recently undergone treatment with an antibiotic. In various embodiments, a subject is to receive one or more antibiotics.

The antibiotics described herein pertain, in various embodiments, to the antibiotic for which there is resistance, or to the antibiotic for which the present beta-lactamase helps induce a therapeutic response.

For example, in various embodiments, the present methods pertain to a regimen in which the present beta-lactamase is administered before the antibiotic (e.g., 1 hour before, or 2 hours before, or 3 hours before, or 6 hours before, or 9 hours before, or 10 hours before, or 12 hours before, or 1 day before) to, without wishing to be bound by theory, modulate the patient's microbiome to be less resistant and therefore responsive to the antibiotic.

In various embodiments, the antibiotic is a selected from beta-lactams, carbapenems, monobactams, β-lactamase inhibitors, aminoglycosides, tetracyclines, rifamycins, macrolides, ketolides, lincosamides, streptogramins, sulphonamides, oxazolidinones, and quinolones.

In various embodiments, the antibiotic is a beta-lactam antibiotic, such as penicillins (e.g., ampicillin, amoxicillin), cephalosporins, clavams (or oxapenams), cephamycins and carbapenems.

In various embodiments, the antibiotic is a cephalosporin, which may be one or more of:

| | |
|---|---|
| Generic | Brand Name |

First Generation

| | |
|---|---|
| Cefacetrile (cephacetrile) | CELOSPOR, CELTOL, CRISTACEF |
| Cefadroxil (cefadroxyl) | DURICEF, ULTRACEF |
| Cefalexin (cephalexin) | KEFLEX, KEFTAB |
| Cefaloglycin (cephaloglycin) | KEFGLYCIN |
| Cefalonium (cephalonium) | |
| Cefaloridine (cephaloradine) | |
| Cefalotin (cephalothin) | KEFLIN |
| Cefapirin (cephapirin) | CEFADYL |
| Cefatrizine | |
| Cefazaflur | |
| Cefazedone | |
| Cefazolin (cephazolin) | ANCEF, KEFZOL |
| Cefradine (cephradine) | VELOSEF |
| Cefroxadine | |
| Ceftezole | |

Second Generation

| | |
|---|---|
| Cefaclor | CECLOR, CECLOR CD, DISTACLOR, |
| | KEFLOR, RANICOR |
| Cefamandole | MANDOL |
| Cefmetazole | |
| Cefonicid | MONOCID |
| Cefotetan | CEFOTAN |
| Cefoxitin | MEFOXIN |
| Cefprozil (cefproxil) | CEFZIL |
| Cefuroxime | CEFTIN, KEFUROX, ZINACEF, |
| | ZINNAT |
| Cefuzonam | |

Third Generation

| | |
|---|---|
| Cefcapene | |
| Cefdaloxime | |
| Cefdinir | OMNICEF, CEFDIEL |
| Cefditoren | SPECTRACEF |
| Cefetamet | |
| Cefixime | SUPRAX |
| Cefmenoxime | CEFMAX |
| Cefodizime | |
| Cefotaxime | CLAFORAN |
| Cefpimizole | |
| Cefpodoxime | VANTIN |
| Cefteram | |
| Ceftibuten | CEDAX |
| Ceftiofur | EXCEDE |
| Ceftiolene | |
| Ceftizoxime | CEFIZOX |
| Ceftriaxone | ROCEPHIN |
| Cefoperazone | CEFOBID |
| Ceftazidime | CEPTAZ, FORTUM, FORTAZ, |
| | TAZICEF, TAZIDIME |

Fourth Generation

| | |
|---|---|
| Cefclidine | |
| Cefepime | MAXIPIME |
| Cefluprenam | |
| Cefoselis | |
| Cefozopran | |
| Cefpirome | CEFROM |
| Cefquinome | |

Fifth Generation

| | |
|---|---|
| Ceftobiprole | ZEFTERA |
| Ceftaroline | TEFLARO |

Not Classified

Cefaclomezine
Cefaloram
Cefaparole
Cefcanel
Cefedrolor
Cefempidone
Cefetrizole
Cefivitril
Cefmatilen
Cefmepidium
Cefovecin
Cefoxazole
Cefrotil
Cefsumide
Cefuracetime
Ceftioxide

In some embodiments, the antibiotic is vancomycin.

In some embodiments, the antibiotic is selected from streptomycin, neomycin, gentamicin, tobramycin, tetracycline, doxycycline, azithromycin, erythromycin, and clarithromycin.

In some embodiments, the antibiotic is selected from trovafloxacin, ciprofloxacin, gatifloxacin, and moxifloxacin.

In some embodiments, the terms “patient” and “subject” are used interchangeably. In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In some embodiments, the subject and/or animal may comprise fluorescently-tagged cells (with e.g., GFP). In some embodiments, the subject and/or animal is a transgenic animal comprising a fluorescent cell.

In various embodiments, methods of the invention are useful in treatment a human subject. In some embodiments, the human is a pediatric human. In other embodiments, the human is an adult human. In other embodiments, the human is a geriatric human. In other embodiments, the human may be referred to as a patient. In some embodiments, the human is a female. In some embodiments, the human is a male.

In certain embodiments, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

Kits

The invention provides kits that can simplify the administration of the modified-release formulation described herein. The kit is an assemblage of materials or components, including at least one of the modified-release formulations described herein. The exact nature of the components configured in the kit depends on its intended purpose. In one embodiment, the kit is configured for the purpose of treating human subjects.

Instructions for use may be included in the kit. Instructions for use typically include a tangible expression describing the technique to be employed in using the components of the kit to affect a desired outcome, such as to treat a disorder associated described herein. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials and components assembled in the kit can be provided to the practitioner store in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label which indicates the contents and/or purpose of the kit and/or its components.

### EXAMPLES

### Example 1: Antibiotic-Mediated Porcine Gut Microbiome Disruption Study Design

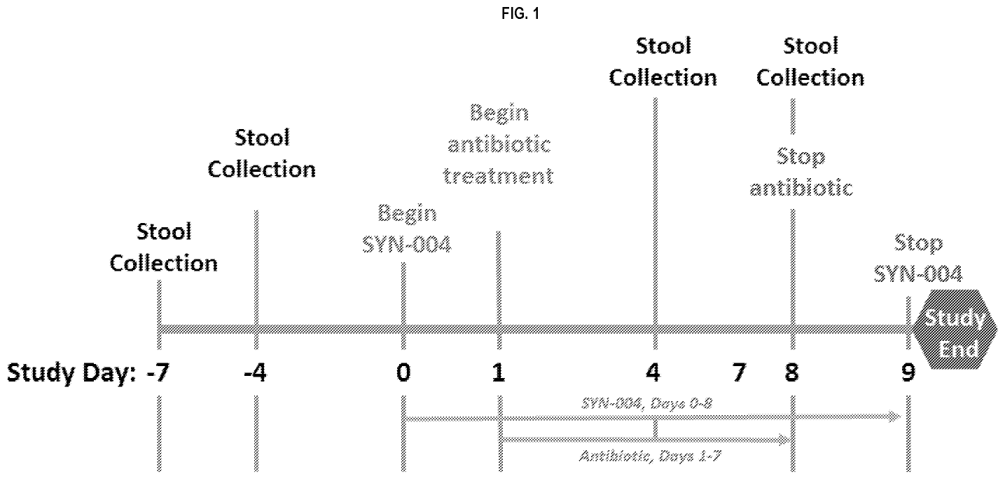

The ability of SYN-004 (ribaxamase) to mitigate the propagation of antibiotic resistance genes caused by exposure of the gut microflora to ceftriaxone was evaluated in normal two month old piglets. See Table 2 and FIG. 1 for study design and timeline. Animals were acclimated for 14 days prior to study initiation. Ceftriaxone (CRO) was administered daily for 7 days and animals that received SYN-004 (ribaxamase) starting the day before antibiotic treatment for 9 consecutive days. Feces were collected on Study Days −7, −4, 4, and 8. Fecal DNA was isolated and subjected to whole genome shotgun metagenomics analyses. The community resistome, the collection of antibiotic resistance genes in the microbiome, was identified by comparison to a curated antibiotic resistance gene database.

TABLE 2
Piglet gut microbiome study design

| | | | SYN-004 |
|---|---|---|---|
| Group (N = 5) | Antibiotic | Antibiotic Delivery | (ribaxamase) |
| 1 | Ceftriaxone | IV, 1x per day | None |
| Pigs 1-5 | (50 mg/kg) | 12 pm | |
| 2 | Ceftriaxone | IV, 1x per day | 1 size 0 capsule |
| Pigs 6-10 | (50 mg/kg) | 12 pm | (75 mg), QID |
| | | | 7am, 12 pm, |
| | | | 5 pm, 10 pm |

Two groups of 5 normal piglets each were used in this study. Group 1 animals were treated with ceftriaxone (IV, 1× per day, 50 mg/kg) for 7 consecutive days and Group 2 animals received ceftriaxone (IV, 1× per day, 50 mg/kg) for 7 consecutive days and SYN-004 (ribaxamase, orally, 4× per day, 75 mg/dose) for 9 consecutive days starting the day before ceftriaxone delivery.

FIG. 1 shows a schematic timeline of pig study. Normal piglets (2 months old, approximately 50 lbs) were acclimated for 14 days prior to study initiation. Two cohorts of 5 animals each were used for this study, Group 1 received ceftriaxone alone and Group 2 received ceftriaxone and SYN-004 (ribaxamase), as indicated in Table 2. Stool was collected at 4 time points, two prior to treatment, at Study Days −7 and −4, and two during treatment at Study Day 4 and 8.

Total DNA was isolated from fecal specimens, using the MOBIO Power-Soil® DNA Isolation Kit (Qiagen, Germantown, MD), following the manufacturer's instructions. Each DNA sample was normalized in 3-18 uL of nuclease-free water for a final concentration of 0.5 ng/uL using the Biomek FX liquid handler (Beckman Coulter Life Sciences, Brea, CA). Libraries were constructed using the Nextera XT Library Prep Kit (illumine, San Diego, CA). For each sample, an input of 0.5 ng was used in the tagmentation reaction, followed by 13 cycles of PCR amplification using Nextera i7 and i5 index primers and 2× KAPA master mix per the modified Nextera XT protocol. The PCR products were purified using 1.0× speed beads and eluted in 15 ul of nuclease-free water. The final libraries were quantified by PicoGreen fluorometric assay (100× final dilution) and the concentrations were in the range of 0.1-4.0 ng/ul. The libraries were pooled, based on concentration determined by PicoGreen and loaded onto a high sensitivity (HS) chip run on the Caliper LabChipGX (Perkin Elmer, Waltham, MA). The base pair size reported was in the range of 301-680 bp. Each pool of 64 was run across 8 lanes of an Illumina HiSeq v3 flowcell, targeting 100 bp paired end reads per sample.

Unassembled metagenomic sequencing reads were directly analyzed using the CosmosID, Inc. bioinformatics software package (CosmosID Inc., Rockville, MD), as described (Hasan et al., 2014, PLoS ONE 9:e97699; Lax et al., 2014, Science 345:1048). Briefly, raw, unassembled shotgun sequence reads were probed against the CosmosID, Inc. curated antibiotic resistance gene database. Analyses of the sequencing data included resistome analyses by identification of antibiotic-resistance genes based on percentage of gene coverage for each gene and quantified by determining the frequency (%) of each gene in each sample and the generation of heatmaps.

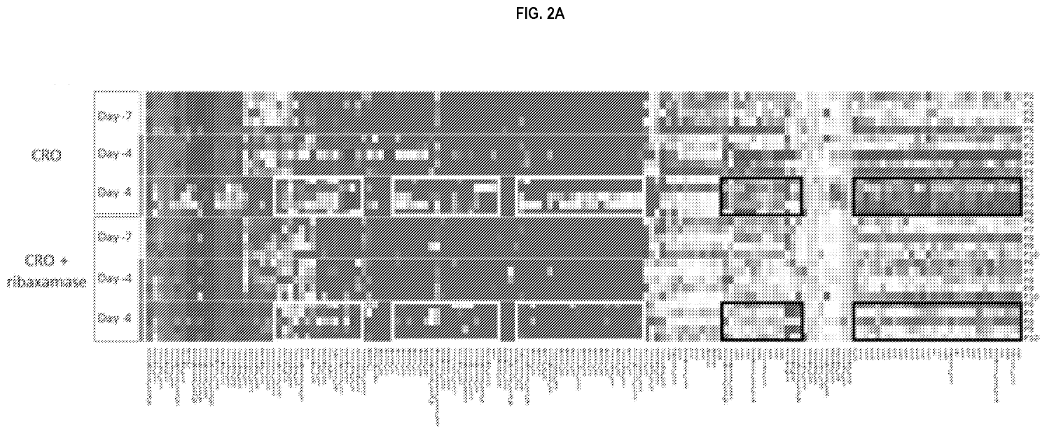

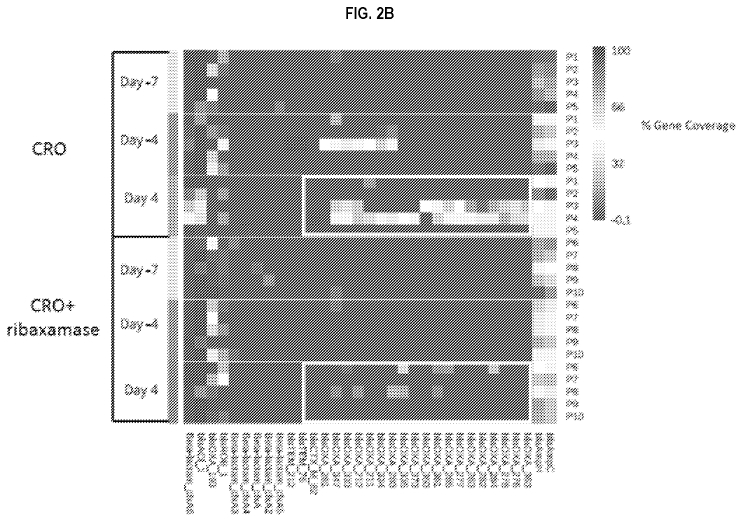

To determine if ribaxamase affected propagation of antibiotic resistance, fecal DNA whole genome shotgun metagenomic data were analyzed for the presence of antibiotic resistant genes, as a measure of the population of antibiotic-resistant bacteria in the fecal microbiomes. Heatmaps of identified antibiotic resistance genes in the fecal microbiome of each animal compared before and after antibiotic treatment (FIG. 2A and FIG. 2B) showed CRO was associated with higher abundance of antibiotic resistance genes post-treatment Day 4 compared to CRO+ribaxamase microbiomes. Most of the resistance genes detected at Day 4 were encoded beta-lactamases (FIG. 2A and FIG. 2B). Specifically, blaOXA genes encoding extended spectrum OXA class D beta-lactamases (McArthur and Write, 2015, Curr Opion Microbiol 27:45; Bush et al., 2016, lahey.org/studies/), were present at high levels in two of five CRO animals at Day 4 but absent or observed at lower frequencies in the CRO+ribaxamase cohort. Similarly, blaCTX-M_82 or bIaCFX_A4 genes, encoding extended spectrum class A serine beta-lactamases (McArthur et al., 2013, Antimicrob Agents Chemother. 57:3348), were detected at Day 4 at high levels in two of the CRO animals. An additional beta-lactamase gene, AmpC, encoding the extended spectrum cephalosporin-resistant class C beta-lactamases, was present in all but one animal prior to antibiotic treatment at Day −7. From Day −7 to Day −4, AmpC levels increased in both cohorts prior to any antibiotic-mediated selection mechanisms. By Day 4, however, AmpC gene frequencies continued to increase in the CRO cohort, while, AmpC decreased to levels similar to the Day −7 frequencies observed in the CRO+ribaxamase animals.

FIG. 2A shows a heat map analysis of the frequency of all antibiotic-resistance genes in the pig fecal microbiomes. Fecal microbiome metagenomic data were analyzed for the presence of antibiotic-resistance genes based on percentage of gene coverage as a measure of the relative gene frequency in each sample. Each row of the heat map represents an individual animal at the indicated time point, Day −7, Day −4, or Day 4. The antibiotic resistance genes are displayed at the bottom of the figure, the treatment group and day of collection of the fecal sample on the left, and the animal numbers on the right (P1-P10).

FIG. 2B shows a heat map analysis of the frequency of beta-lactamase genes in the pig fecal microbiomes. Fecal microbiome metagenomic data were analyzed for the presence of beta-lactamase genes based on percentage of gene coverage as a measure of the relative gene frequency in each sample. Each row of the heat map represents an individual animal at the indicated time point, Day −7, Day −4, or Day 4. The antibiotic resistance genes are displayed at the bottom of the figure, the treatment group and day of collection of the fecal sample on the left, and the animal numbers on the right (P1-P10).

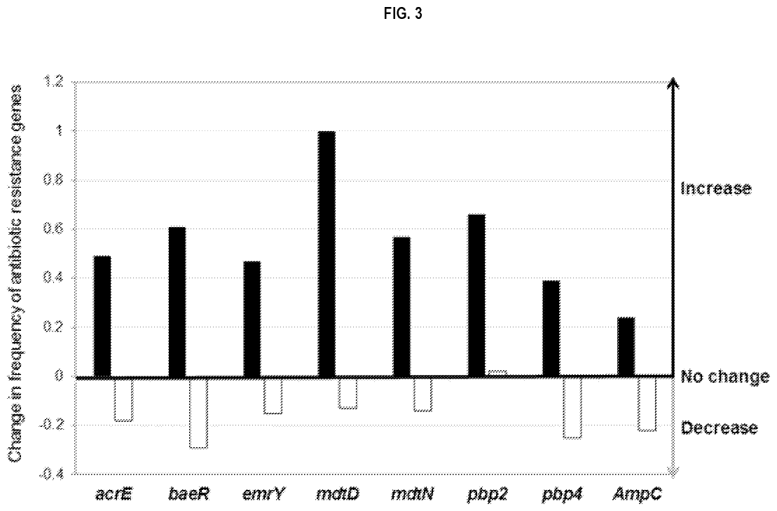

In addition to beta-lactamases, other resistance genes displayed an increased frequency in response to antibiotic exposure. Many of these genes encode components of multidrug efflux transporter systems, systems that confer resistance to a broad range of antibiotics, including the beta-lactams. The genes selected for additional analyses include: acrE, encoding a component of the AcrEF-ToIC multidrug efflux transporter system (Lau and Zgurskaya, 2005, J. Bacteriol. 187:7815); baeR; encoding a response regulator of the MdtABC multidrug efflux transporter system (Nagakubo et al., 2002, J. Bacteriol. 184:4161); emrY, encoding a component of the EmrKY-ToIC multidrug efflux transporter system (Tanabe et al., 1997, J. Gen. Appl. Microbiol. 43:257); mdtD, encoding a component of the MdtABC multidrug efflux transporter system (Nagakubo et al., 2002, J. Bacteriol. 184:4161); and mdtN, encoding a multidrug resistance efflux pump from the major facilitator superfamily (Sulavik et al., 2001, Antimicrob. Agents Chemother. 45:1126). Two genes closely related to the beta-lactamases, pbp2, encoding penicillin binding protein 2 (Bharat et al., 2015, Antimicrob. Agents Chemother. 59:5003), and pbp4, encoding penicillin binding protein 4 (Sun et al., 2014, PLoS One 9:e97202) were also chosen, in addition to the beta-lactamase, AmpC, encoding the cephalosporin-resistant Class C beta-lactamases. For each selected gene, change in the mean of relative gene frequencies from Day −4 to Day 4 was compared in the CRO or the CRO+ribaxamase cohorts (FIG. 3). Relative gene frequencies increased for each gene in the CRO cohort. In contrast, the gene frequencies decreased for each gene in the CRO+ribaxamase group except for pbp2, whose levels increased only slightly above baseline. The greatest increase was observed for the mdtD gene, whose levels doubled from Day −4 to Day 4 in the CRO cohort, while mdtD levels were reduced in the CRO+ribaxamase cohort.

FIG. 3 shows changes in the frequency of selected antibiotic resistance genes. The change in the relative frequency (mean) of the indicated antibiotic resistance genes for the CRO (black) or CRO+ribaxamase (white) treated animals from pretreatment Day −4 compared to post-treatment Day 4 is displayed. A negative value indicates a reduction in frequency, a positive value indicates an increased frequency, and a zero value represents no change in gene frequency. The genes are listed on the horizontal axis: acrE, encodes a component of the AcrEF-ToIC multidrug efflux transporter; baeR; encodes a response regulator of the MdtABC multidrug efflux transporter system; emrY, encodes a component of the EmrKY-ToIC multidrug efflux transporter system; mdtD, encodes a component of the MdtABC multidrug efflux transporter system; mdtN, encodes a multidrug resistance efflux pump from the major facilitator superfamily; pbp2, encodes penicillin binding protein 2; pbp4, encodes penicillin binding protein 4; and AmpC, encodes a class C beta-lactamase.

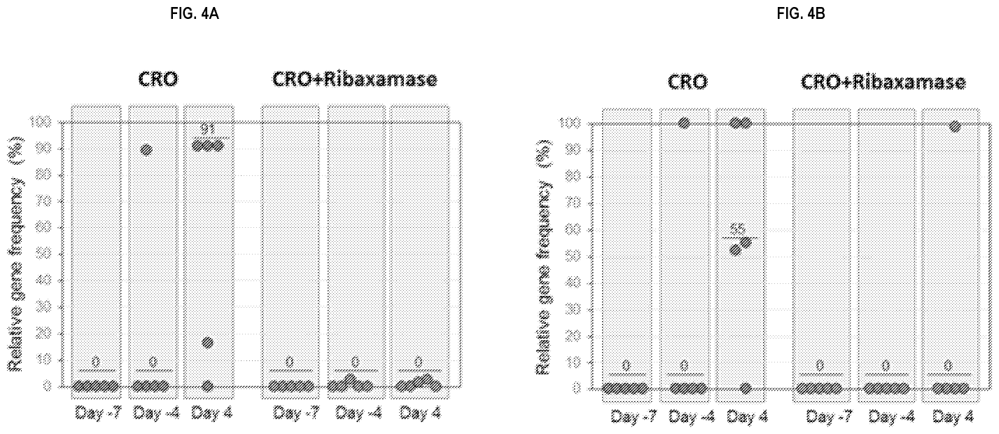

Two antibiotic-resistance genes that convey resistance to non-beta-lactam antibiotics, aminoglycosides and tetracycline, were selected for further analysis (FIG. 4A and FIG. 4B). Aminoglycoside_strA (Scholz et al., 1989, Gene 75:271) encodes an aminoglycoside phosphotransferase, and Tetracycline_tet39 (Agerso and Guardabassi, 2005, J. Antimicrob. Chemother. 55:566) encodes a component of a tetracycline efflux pump. Neither gene was detected in the pretreatment samples (Day −7). At Day −4 both genes were detected in the one of the animals (Pig 3). At day 4 after antibiotic treatment, both genes were detected at high levels in 4 of 5 animals treated with CRO. In contrast, in the CRO+ribaxamase cohort, aminoglycoside_strA levels remained low or absent in all animals and only one animal in the CRO+ribaxamase cohort was identified with a high level the tetracycline_tet39 gene.

FIG. 4A and FIG. 4B show changes in the frequency of selected antibiotic resistance genes. The relative frequency of two selected antibiotic-resistance genes, A) Aminoglycoside_strA and B) Tetracycline_tet39, are displayed from CRO (blue) or CRO+ribaxamase (orange) treated animals. Each dot represents the relative gene level in each animal's microbiome from pretreatment Days −7, or −4, or post-treatment Day 4. The medians are displayed in each data set.

Ceftriaxone treatment increased the abundance of antibiotic resistance genes in the porcine gut microbiome while SYN-004 (ribaxamase) attenuated this enrichment process. In the presence of ribaxamase, antibiotic resistance gene frequencies were reduced or maintained at pre-antibiotic treatment levels. Notably, in the presence of ceftriaxone, the frequencies of many classes of antibiotic resistance genes were amplified, not solely genes that conferred resistance to the beta-lactams. These antibiotic resistance genes included beta-lactamases, genes encoding components of drug efflux pump systems, and genes encoding resistances to tetracycline and aminoglycosides.

### Example 2: Extension of Antibiotic-Mediated Porcine Gut Microbiome Disruption Study Design

The study of Example 1 was extended to oral amoxicillin and IV ertapenem.

TABLE 3
Piglet gut microbiome study design

| Group (n = 5) | Antibiotic | Antibiotic Delivery |
|---|---|---|
| 1 | Amoxicillin | Oral, BID, each dose 20 mg/kg |
| Pigs 1, 2, 3, 5, and 5 | | 7 am, 5 pm |
| 2 | Ertapenem | IV, 1x per day, 30 mg/kg, |
| Pigs 6, 7, 8, 9, 10 | | 12 pm |

Approximately 2 month old Yorkshire piglets, approx. 20 kg were used. Pigs received 2 ml of Iron/Penicillin/Draxxin on day of birth and again at 7 days old. Antibiotics were not provided in food. Pigs were weaned at 21 days. Piglets were fed 3× a day, after dosing at 7 am, after dosing at 12 pm, after dosing at 5 pm.

Whole genome shotgun sequence analyses of pig fecal DNA were performed to assess the effect of the antibiotics on the gut microbiota and quantify antibiotic-resistance genes as a measure of antibiotic-resistant bacteria present in the fecal microbiome. Both antibiotics caused significant changes to the gut microbiome.

Within 4 days of antibiotic exposure, a broad spectrum of resistance genes, including those conferring resistance to beta-lactam and non-beta-lactam antibiotics, was detected in the microbiomes.

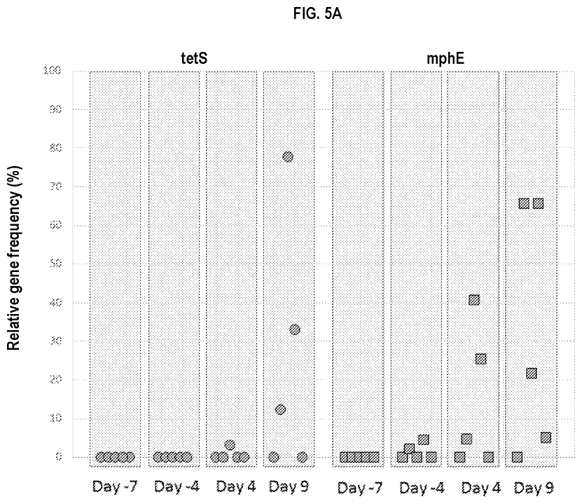

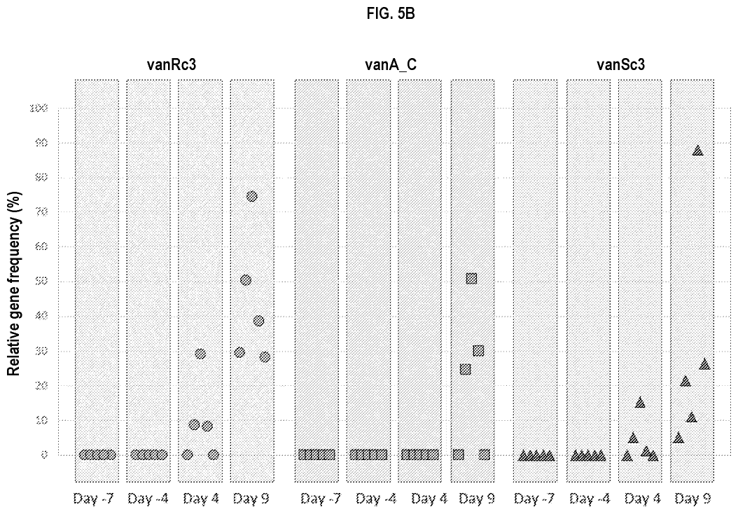

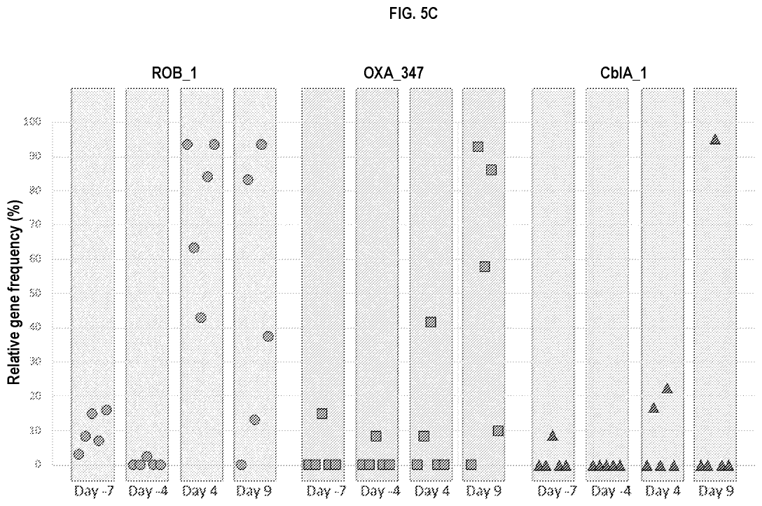

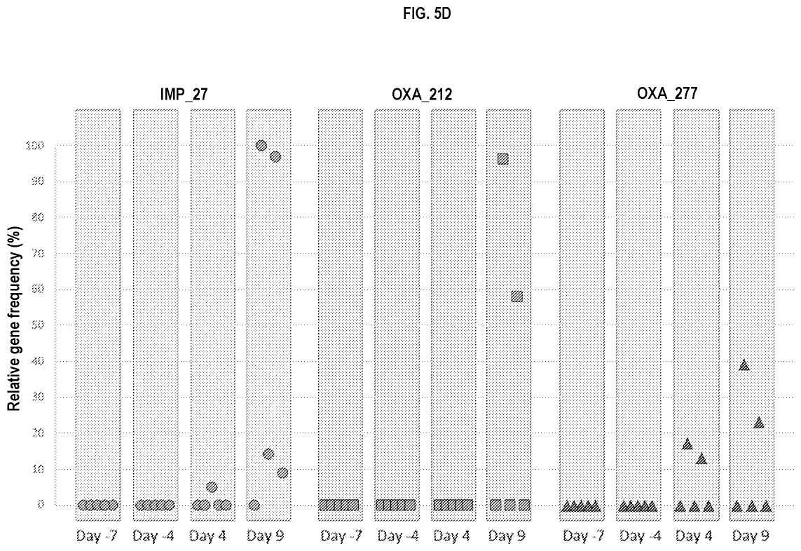

FIG. 5A shows changes in the frequency of antibiotic resistance genes tetS and mphE (shown left to right) upon ertapenem treatment.

FIG. 5B shows changes in the frequency of vancomycin resistance genes vanRc3, vanA_C, and vanSc3 (shown left to right) upon ertapenem treatment.

FIG. 5C shows changes in the frequency of beta-lactamase genes ROB_1, OXA_347, and CbIA_1 (shown left to right) upon amoxicillin treatment.

FIG. 5D shows changes in the frequency of beta-lactamase genes IMP_27, OXA_212, and OXA_277 upon ertapenem treatment.

It is anticipated that SYN-004 will also mitigate emergence and spread of antibiotic resistance when a subject is administered oral amoxicillin and IV ceftriaxone, consistent with results of IV ceftriaxone above.

### Example 3 Oral Antibiotic-Mediated Canine Gut Microbiome Disruption Study Design

The ability of a delayed-release formulation of SYN-004 (ribaxamase), to mitigate the propagation of antibiotic resistance genes caused by exposure of the gut microbiota to oral amoxicillin was evaluated in normal, seven month old female beagles. Animals were acclimated for 24 days prior to dosing initiation. Animals (n=10, 5 animals per cohort) received oral amoxicillin (40 mg/kg/dose, reconstituted in apple juice) three times a day+/−one capsule of SYN-004 (10 mg) for 5 days, with the last dose on the morning of day 6. The animals received 16 total doses of amoxicillin+/−SYN-004. Fecal samples were collected prior to antibiotic dosing and after the last dose. Blood was collected after the first antibiotic dose on day 1 and after the last dose on day 6. On each blood collection day, blood was collected from each animal at seven time points, 0.5, 1, 2, 3, 4, 6, and 8 hrs. Amoxicillin serum levels were measured using a validated liquid chromatography (LC) method with tandem mass spectrometry detection (LC/MS/MS). DNA isolated from fecal samples was subjected to whole genome shotgun sequencing and metagenomics analyses.

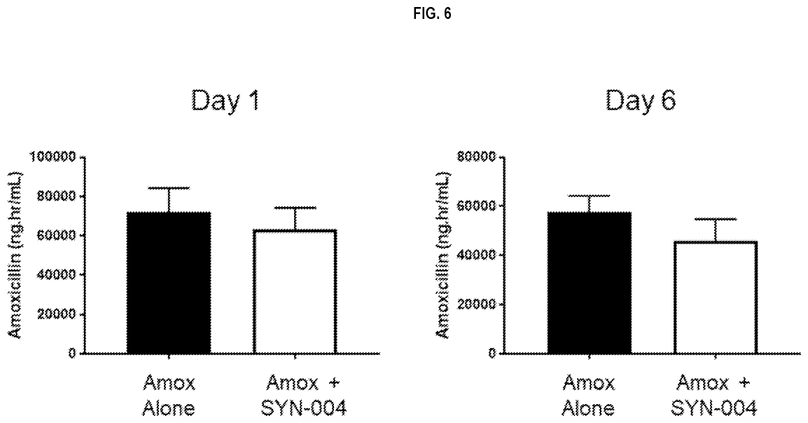

Amoxicillin serum levels were plotted for each group for each blood collection day as mean+standard deviation for each time point. Area under the curve was calculated for each group for each bleed day (day 1 and day 6) and were compared statistically using one-way ANOVA with Dunnett's multiple comparison tests (Graph Pad Prism 7.03 software). Amoxicillin serum levels were not significantly different for the amoxicillin alone and the amoxicillin+SYN-004 cohorts at day 1 and day 6, p=0.703 and 0.098, respectively (FIG. 6). These data demonstrate that the delayed release SYN-004 formulation did not interfere with amoxicillin systemic absorption from the gastrointestinal tract and indicate that this SYN-004 formulation was not released prematurely prior to amoxicillin absorption in the upper small intestine in the dog.

DNA isolated from the fecal samples was subjected to whole genome sequencing and analyzed using the CosmosID, Inc. bioinformatics software package. Analyses included identification of the microbiota species present in the fecal microbiomes and identification of antibiotic-resistance genes based on the percentage gene coverage for each gene and quantified by determining the frequency (%) of each gene in each sample.

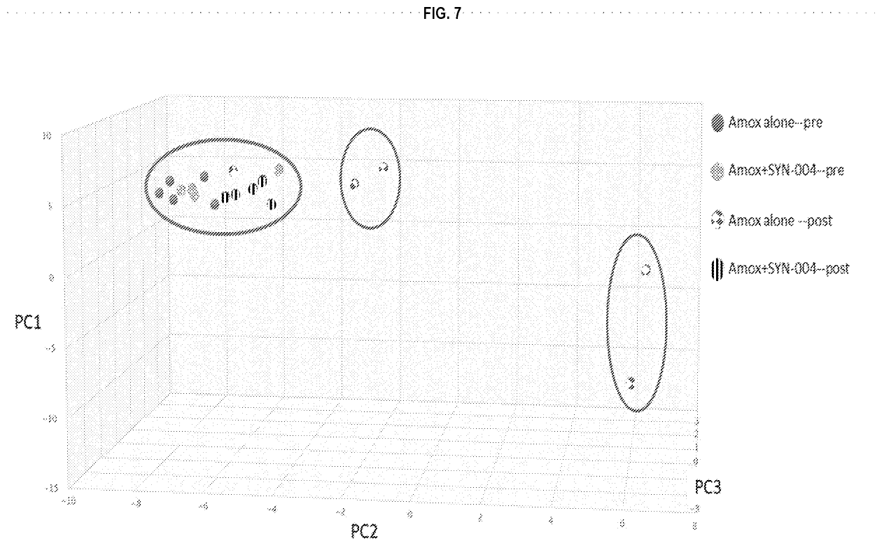

To determine if SYN-004 protected the fecal microbiomes from damage caused by oral amoxicillin, the microbiome bacterial populations were compared using a three coordinate Principal Coordinate Analysis (CosmosID, Inc. bioinformatics software). Distance between points indicates degree of difference in sample diversity with points close together more similar. The pre-treatment microbiomes from both the amoxicillin alone and amoxicillin+SYN-004 cohorts clustered together (FIG. 7). Notably, the post-amoxicillin exposure microbiomes from the amoxicillin+SYN-004 cohort also clustered with the pretreatment samples. In contrast, 4/5 post treatment samples were plotted far from the pretreatment cluster. One post treatment amoxicillin alone sample was clustered together with the rest of the samples. These data demonstrate that oral amoxicillin disrupts the microbiota populations in the GI tract and that SYN-004 can mitigate the damage caused by oral amoxicillin.

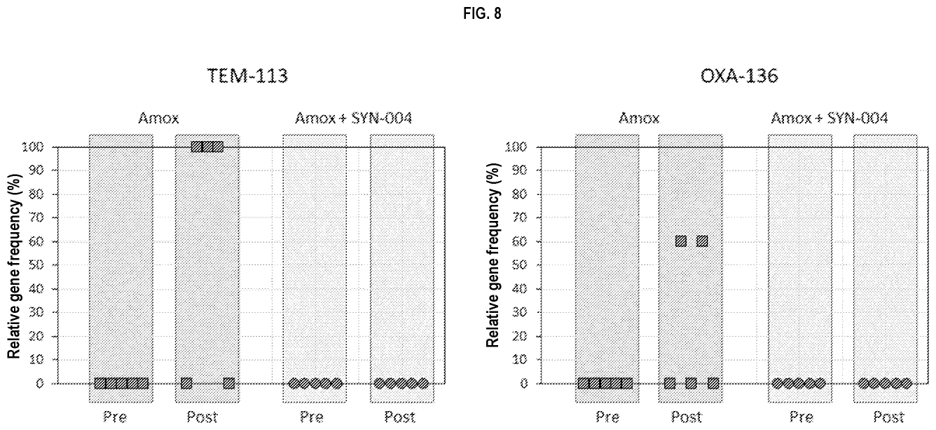

To determine if SYN-004 affected the abundance of antibiotic resistance genes, resistome analyses were performed. Comparison of the frequency of selected antibiotic resistance genes pretreatment to post treatment demonstrated that amoxicillin was associated with higher abundance of antibiotic resistance genes post treatment compared to the amoxicillin+SYN-004 cohort. Many of the resistance genes detected post treatment were encoded beta-lactamases. Notably, several TEM and OXA beta-lactamase genes were detected only at day 6 in the amoxicillin alone cohort (FIG. 8). These genes were not detected prior to amoxicillin exposure.

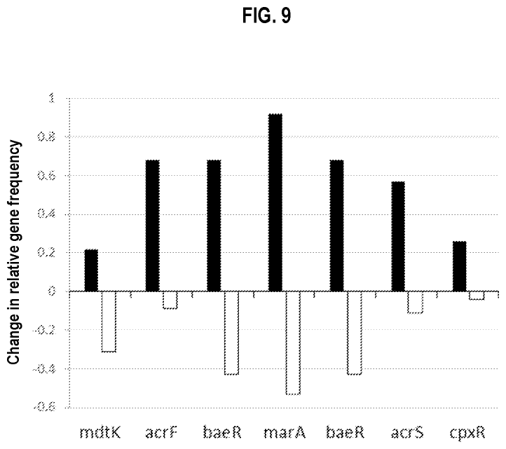

In addition to beta-lactamases, other resistance genes displayed an increased frequency in response to antibiotic exposure. Many of these genes encoded multidrug efflux transporter system components, systems that confer resistance to a broad range of antibiotics, including the beta-lactams. Several genes were selected for further analysis. For each selected gene, the change in the mean of relative gene frequencies from pretreatment to post treatment was compared in the amoxicillin alone or the amoxicillin+SYN-004 cohorts (FIG. 9). Relative gene frequencies increased for each gene in the amoxicillin alone cohort. In contrast, the gene frequencies decreased for each gene in the amoxicillin+SYN-004 cohort. The greatest increase was observed for the marA gene, whose levels almost doubled from pretreatment to post treatment, while marA levels were reduced by approximately 50% in the amoxicillin+SYN-004 cohort.

Oral amoxicillin exposure increased the abundance of antibiotic resistance genes in the canine gut microbiome while SYN-004 attenuated this enrichment process. In the presence of SYN-004, antibiotic resistance gene frequencies were reduced. Notably, in the presence of amoxicillin, the frequencies of many classes of antibiotic resistance genes were amplified, not solely genes that conferred resistance to the beta-lactams. These antibiotic resistance genes included beta-lactamases, genes encoding components of drug efflux pump systems, and genes encoding resistances to macrolides and aminoglycosides.

### Example 4: Mitigation of Antibiotic Resistance Gene Emergence and Propagation Following IV Carbapenem Treatment

The ability of the carbapenemase, P2A, to mitigate the propagation of antibiotic resistance genes caused by exposure of the gut microbiota to the carbapenem antibiotic, ertapenem, is evaluated in normal, two month old pigs. Animals are acclimated for 24 days prior to dosing initiation. Animals (n=16, 8 animals per cohort) receive ertapenem (30 mg/kg/dose, IV, once a day for 4 days)+/−one capsule of P2A (50 mg, PO, 4 times a day). The P2A is started the day before antibiotic treatment and continues with one final dose the morning of day 5. Fecal samples are collected prior to antibiotic dosing and after the last dose of P2A on day 5. Blood is collected on day 3 after 3 ertapenem doses. Blood is collected at 3 timepoints, 1 hr, 2 hrs, and 3 hrs after ertapenem. Ertapenem serum levels are measured using a validated liquid chromatography (LC) method with tandem mass spectrometry detection (LC/MS/MS). DNA is isolated from fecal samples and is subjected to whole genome shotgun sequencing and metagenomics analyses.

Ertapenem serum levels are not expected to be different in the two cohorts, ertapenem alone or ertapenem+P2A. DNA isolated from the fecal samples is subjected to whole genome shotgun sequencing and is analyzed using the CosmosID, Inc. bioinformatics software package. Analyses include identification of the microbiota species present in the fecal microbiomes and identification of antibiotic-resistance genes based on the percentage gene coverage for each gene and are quantified by determining the frequency (%) of each gene in each sample.

Microbiome analyses, including Principal Coordinate Analysis, heatmap analysis, and/or additional metagenomics analyses are expected to demonstrate that ertapenem alone causes disruption to the microbiome by changing the microbiota composition. In contrast, ertapenem+P2A protects the microbiome and mitigates microbiome disruption.

Resistome analyses, including heatmap analysis, Principal Coordinate Analysis, and/or additional gene analyses are expected to demonstrate that ertapenem alone causes a broad range of antibiotic resistance genes to propagate and emerge. In contrast, emergence and propagation of antibiotic resistance genes is mitigated in the presence of P2A.

These data are expected to demonstrate that IV ertapenem disrupts the microbiota populations in the GI tract and that P2A mitigates the damage caused by IV ertapenem and protects the gut microbiome. In addition, resistome analyses are expected to demonstrate that ertapenem causes the emergence of a broad range of antibiotic resistance genes, including those that confer resistance to carbapenems and other beta-lactam antibiotics, as well others that confer resistance to other antibiotic classes, and that P2A mitigates the emergence and propagation of antibiotic resistance genes.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50%” covers the range of 45% to 55%.

An “effective amount,” when used in connection with medical uses is an amount that is effective for providing a measurable treatment, prevention, or reduction in the rate of pathogenesis of a disorder of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents (e.g., beta-lactamases and/or additional therapeutic agents described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures, tissue samples, tissue homogenates or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### REFERENCES

The following references are hereby incorporated by reference in their entireties
Hasan N A, Young B A, Minard-Smith A T, Saeed K, Li H, Heizer E M, McMlllan M J, Isom R, Abdullah, A S, Bornman D M, Faith S A, Choi S A, Dickens M L, Cebula T A, Colwell R R. (2014). Microbial community profiling of human saliva using shotgun metagenomics sequencing. PLoS ONE 9(5):e97699. Doi:10.1371/journal.pone.0097699.
Lax S, Smith D P, Marcell J H, Owens S, Handley K, Scott K, Gibbons S, Larsen P, Shogan B D, Weiss S, Metcalf J K, Ursell L K, Vazquez-Baeza Y, Treuren V W, Hasan N A, Gibson M K, Colwell R R, Dantas G, Knight R, Gilbert J A. (2014). Longitudinal analysis of microbial interaction between humans and the indoor environment. Science 345, 1048 (2014); DOI:1 126/science.1254529.
McArthur A G, Wright G D. 2015. Bioinformatics of antimicrobial resistance in the age of molecular epidemiology. Curr Opin Microbiol 27:45-50.
Bush K, Palzkill, T., Jacoby, G. 2016. Beta-lactamase classification and amino acid sequences for TEM, SHV and OXA Extended-Spectrum and Inhibitor Resistant Enzymes. lahey.org/studies/. Accessed September 6.
McArthur A G, Waglechner N, Nizam F, Yan A, Azad M A, Baylay A J, Bhullar K, Canova M J, De Pascale G, Ejim L, Kalan L, King A M, Koteva K, Morar M, Mulvey M R, O'Brien J S, Pawlowski A C, Piddock L J, Spanogiannopoulos P, Sutherland A D, Tang I, Taylor P L, Thaker M, Wang W, Yan M, Yu T, Wright G D. 2013. The comprehensive antibiotic resistance database. Antimicrob Agents Chemother 57:3348-3357.
Lau S Y, Zgurskaya H I. 2005. Cell division defects in Escherichia coli deficient in the multidrug efflux transporter AcrEF-ToIC. J Bacteriol 187:7815-7825.
Nagakubo S, Nishino K, Hirata T, Yamaguchi A. 2002. The putative response regulator BaeR stimulates multidrug resistance of Escherichia coli via a novel multidrug exporter system, MdtABC. J Bacteriol 184:4161-4167.
Tanabe H, Yamasak K, Furue M, Yamamoto K, Katoh A, Yamamoto M, Yoshioka S, Tagami H, Aiba H A, Utsumi R. 1997. Growth phase-dependent transcription of emrKY, a homolog of multidrug efflux emrAB genes of Escherichia coli, is induced by tetracycline. J Gen Appl Microbiol 43:257-263.
Sulavik M C, Houseweart C, Cramer C, Jiwani N, Murgolo N, Greene J, DiDomenico B, Shaw K J, Miller G H, Hare R, Shimer G. 2001. Antibiotic susceptibility profiles of Escherichia coli strains lacking multidrug efflux pump genes. Antimicrob Agents Chemother 45:1126-1136.
Bharat A, Demczuk W, Martin I, Mulvey M R. 2015. Effect of Variants of Penicillin-Binding Protein 2 on Cephalosporin and Carbapenem Susceptibilities in Neisseria gonorrhoeae. Antimicrob Agents Chemother 59:5003-5006.
Sun S, Selmer M, Andersson D I. 2014. Resistance to beta-lactam antibiotics conferred by point mutations in penicillin-binding proteins PBP3, PBP4 and PBP6 in Salmonella enterica. PLoS One 9:e97202.
Scholz P, Haring V, Wittmann-Liebold B, Ashman K, Bagdasarian M, Scherzinger E. 1989. Complete nucleotide sequence and gene organization of the broad-host-range plasmid RSF1010. Gene 75:271-288.
Agerso Y, Guardabassi L. 2005. Identification of Tet 39, a novel class of tetracycline resistance determinant in Acinetobacter spp. of environmental and clinical origin. J Antimicrob Chemother 55:566-569.

1.-30. (canceled)

31. A method for mitigating emergence or spread of an antibiotic resistance in a patient in need thereof, comprising administering an effective amount of a beta-lactamase having an amino acid sequence of at least 95% identity with SEQ ID NO: 1 and a hydrophilic residue other than aspartate (D) at position 276 based on Ambler classification, wherein the beta-lactamase is administered before or concurrently with an antibiotic.

32. The method of claim 31, wherein the method preserves the resistome state of the subject before administration of the antibiotic.

33. The method of claim 32, wherein the method reduces or prevents an increased resistome presence in the subject.

34. The method of claim 31, wherein the antibiotic resistance comprises one or more of aminoglycoside resistance, tetracycline resistance, carbapenem resistance, and vancomycin resistance.

35. The method of claim 31, wherein the antibiotic resistance is characterized by an increase in the frequency of one or more genes listed in Table A.

36. The method of claim 35, wherein the antibiotic resistance is characterized by an increase in the frequency of one or more genes selected from strA, tet39, tetS, mphE, ROB_1, OXA_347, CblA_1, IMP_27, OXA_212, OXA_277, vanRc3, vanA_C, vanSc3, mdtK, acrF, baeR, marA, acrS, TEM-113, OXA-136, and cpxR.

37. The method of claim 31, wherein the antibiotic is selected from a beta-lactam, a beta-lactamase inhibitor, a penicillin, a monobactam, and a cephalosporin.

38. The method of claim 37, wherein the antibiotic is ceftriaxone, cefotaxime, cefazolin, cefoperazone, cefuroxime, cefacetrile (cephacetrile), cefepime, cefadroxil (cefadroxyl), cefalexin (cephalexin), cefaloglycin (cephaloglycin), cefalonium (cephalonium), cefaloridine (cephaloradine), cefalotin (cephalothin), cefapirin (cephapirin), cefatrizine, cefazaflur, cefazedone, cefradine (cephradine), cefroxadine, ceftezole, cefaclor, cefamandole, cefmetazole, cefonicid, cefotetan, cefoxitin, cefprozil (cefproxil), cefuzonam, cefcapene, cefdaloxime, cefdinir, cefditoren, cefetamet, cefixime, cefmenoxime, cefodizime, cefpimizole, cefpodoxime, cefteram, ceftibuten, ceftiofur, ceftiolene, ceftizoxime, ceftazidime, cefclidine, cefluprenam, cefoselis, cefozopran, cefpirome, cefquinome, ceftobiprole, ceftaroline, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, cefuracetime, ceftioxide, amoxicillin, piperacillin, ampicillin, or penicillin.

39. The method of claim 31, wherein the beta-lactamase has an amino acid sequence of at least 97% identity with SEQ ID NO: 1.

40. The method of claim 31, wherein the beta-lactamase has an amino acid sequence of at least 98% identity with SEQ ID NO: 1.

41. The method of claim 31, wherein the beta-lactamase has an amino acid sequence of at least 99% identity with SEQ ID NO: 1.

42. The method of claim 31, wherein the patient has or is at risk for a bloodstream infection (BSI), catheter or intravascular-line infections, central-line infections, chronic inflammatory diseases, meningitis, pneumonia, ventilator-associated pneumonia, skin or soft tissue infections, surgical-site infections, urinary tract infections, antibiotic-resistant urinary tract infections, catheter-associated urinary tract infections, wound infections, antibiotic-resistant infections or antibiotic-sensitive infections.

43. The method of claim 31, wherein the method prevents an infection.

44. The method of claim 43, wherein the infection is antibiotic-resistant proteobacteria, vancomycin resistant enterococcus (VRE), carbapenem resistant enterobacteriaceae (CRE), fluoroquinolone-resistant enterobacteriaceae, or extended spectrum beta-lactamase producing enterobacteriaceae (ESBL-E).

45. The method of claim 43, wherein the infection is a C. difficile infection (CDI) and/or a C. difficile-associated disease.


SEQUENCE LISTING (6 sequences)

SEQ ID NO: 1 - protein, 264 aa, synthetic construct
  REGION 1..264: note = Synthetic Sequence
TEMKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVADKTGAASYGTRNDIAIIWPPKGDPVVLAVLSSRDKKDAKYDNKLIAEATKVVMKALNMNGK

SEQ ID NO: 2 - DNA, 795 bp, synthetic construct
  misc_feature 1..795: note = Synthetic Sequence
  source 1..795: mol_type = other DNA
atgactgagatgaaagatgattttgcgaagctggaagaacagtttgacgcaaaattgggcattttcgcgttggacacgggtacgaatcgtacggttgcctaccgtccggacgagcgcttcgccttcgcgagcacgatcaaagccctgaccgtcggcgtgctgctccagcaaaagagcatcgaggacctgaaccagcgcattacctacacccgtgatgatctggtgaactataatccgatcaccgagaaacacgttgataccggtatgaccctgaaagaactggcagatgcaagcctgcgctacagcgataacgcggctcagaatctgattctgaagcaaatcggtggtccggagagcttgaagaaagaactgcgtaaaatcggcgatgaagtcactaatccggagcgttttgagccggagctgaacgaagtgaatccgggtgaaacgcaagacacgagcaccgcgcgtgcgcttgtcacctccctgcgcgctttcgcactggaagataagctgccgtcggagaaacgcgagctgctgatcgactggatgaagcgcaatacgaccggcgacgcgctgattcgtgcgggcgttccggacggttgggaagtggctgacaagaccggtgcggcgagctacggcacccgtaacgatatcgcgatcatttggccacctaaaggtgacccggtcgtgctggccgtactgagcagccgtgacaagaaagacgcaaagtatgataacaagctgattgcagaggcgaccaaagttgttatgaaggcactgaacatgaatggtaag

SEQ ID NO: 3 - protein, 262 aa, synthetic construct
  REGION 1..262: note = Synthetic Sequence
EMKDDFAKLEEQFDAKLGIFALDTGTNRTVAYRPDERFAFASTIKALTVGVLLQQKSIEDLNQRITTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVGDKTGSGDYGTRNDIAIIWPPKGDPVVLAVLSSRDKKDAKYDNKLIAEATKVVMKALNMNGK

SEQ ID NO: 4 - protein, 299 aa, synthetic construct
  REGION 1..299: note = Synthetic Sequence
MIQKRKRTVSFRLVLMCTLLFVSLPITKTSAQASKTEMKDDFAKLEEQFDAKLGIFALDTGTNRTVAYRPDERFAFASTIKALTVGVLLQQKSIEDLNQRITYTRDDLVNYNPITEKHVDTGMTLKELADASLRYSDNAAQNLILKQIGGPESLKKELRKIGDEVTNPERFEPELNEVNPGETQDTSTARALVTSLRAFALEDKLPSEKRELLIDWMKRNTTGDALIRAGVPDGWEVGDKTGSGDYGTRNDIAIIWPPKGDPVVLAVLSSRDKKDAKYDNKLIAEATKVVMKALNMNGK

SEQ ID NO: 5 - DNA, 900 bp, synthetic construct
  misc_feature 1..900: note = Synthetic Sequence
  source 1..900: mol_type = other DNA
atgattcaaaaacgaaagcggacagtttcgttcagacttgtgcttatgtgcacgctgttatttgtcagtttgccgattacaaaaacatcagcgcaagcttccaagacggagatgaaagatgattttgcaaaacttgaggaacaatttgatgcaaaactcgggatctttgcattggatacaggtacaaaccggacggtagcgtatcggccggatgagcgttttgcttttgcttcgacgattaaggctttaactgtaggcgtgcttttgcaacagaaatcaatagaagatctgaaccagagaataacatatacacgtgatgatcttgtaaactacaacccgattacggaaaagcacgttgatacgggaatgacgctcaaagagcttgcggatgcttcgcttcgatatagtgacaatgcggcacagaatctcattcttaaacaaattggcggacctgaaagtttgaaaaaggaactgaggaagattggtgatgaggttacaaatcccgaacgattcgaaccagagttaaatgaagtgaatccgggtgaaactcaggataccagtacagcaagagcacttgtcacaagccttcgagcctttgctcttgaagataaacttccaagtgaaaaacgcgagcttttaatcgattggatgaaacgaaataccactggagacgccttaatccgtgccggtgtgccggacggttgggaagtgggtgataaaactggaagcggagattatggaacccggaatgacattgccatcatttggccgccaaaaggagatcctgtcgttcttgcagtattatccagcagggataaaaaggacgccaagtatgataataaacttattgcagaggcaacaaaggtggtaatgaaagccttaaacatgaacggcaaataa

SEQ ID NO: 6 - protein, 216 aa, synthetic construct
  REGION 1..216: note = Synthetic Sequence
ETGTISISQLNKNVWVHTELGYFNGEAVPSNGLVLNTSKGLVLVDSSWDNKLTKELIEMVEKKFQKRVTDVIITHAHADRIGGITALKERGIKAHSTALTAELAKNSGYEEPLGDLQTITSLKFGNTKVETFYPGKGHTEDNIVVWLPQYQILAGGCLVKSAEAKDLGNVADAYVNEWSTSIENVLKRYGNINSVVPGHGEVGDKGLLLHTLDLLK